SEQUENCE LISTING (15 sequences)

SEQ ID NO: 1 - protein, 448 aa, synthetic construct
  REGION 1..448: note = H1L1 Heavy Chain
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 214 aa, synthetic construct
  REGION 1..214: note = H1L1 Light Chain
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 119 aa, synthetic construct
  REGION 1..119: note = H1L1 VH
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSS

SEQ ID NO: 4 - protein, 107 aa, synthetic construct
  REGION 1..107: note = H1L1 VL
DIQMTQSPATLSVSPGERATLSCKASQNVGGNVDWYQWKPGQAPRLLIYGASNRYTGIPARFRGSGSGTEFTLTISSLQSEDFAVYYCLQYKNNPWTFGQGTKVEIK

SEQ ID NO: 5 - protein, 8 aa, synthetic construct
  REGION 1..8: note = CDR H1
GFSFSSYG

SEQ ID NO: 6 - protein, 8 aa, synthetic construct
  REGION 1..8: note = CDR H2
ISPSGSYF

SEQ ID NO: 7 - protein, 9 aa, synthetic construct
  REGION 1..9: note = CDR H3
PGFNPPFDH

SEQ ID NO: 8 - protein, 6 aa, synthetic construct
  REGION 1..6: note = CDR L1
QNVGGN

SEQ ID NO: 9 - skipped

SEQ ID NO: 10 - protein, 9 aa, synthetic construct
  REGION 1..9: note = CDR L3
LQYKNNPWT

SEQ ID NO: 11 - protein, 448 aa, synthetic construct
  REGION 1..448: note = H3L3 Heavy Chain
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 214 aa, synthetic construct
  REGION 1..214: note = H3L3 Light Chain
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 119 aa, synthetic construct
  REGION 1..119: note = H3L3 VH
QVQLVESGGGVVQPGRSLRLSCLGSGFSFSSYGMNWVRQAPGKGLEWVASISPSGSYFYYADSVKGRFTISRDNSKNTLYLQMTSLRAEDTAIYYCARLPGFNPPFDHWGQGTLVTVSS

SEQ ID NO: 14 - protein, 107 aa, synthetic construct
  REGION 1..107: note = H3L3 VL
DIQMTQSPSSLSASVGDRVTITCKASQNVGGNVDWYQWKPGKAPKLLIYGASNRYTGVPDRFRGSGSGTDFTLTISSLQPEDVATYYCLQYKNNPWTFGGGTKVEIK

SEQ ID NO: 15 - protein, 448 aa, synthetic construct
EVQLVESGGGLVKPGGSLRLSCAASGFSFSSYGMNWVRQAPGKGLEWVSSISPSGSYFYYADSVKGRFTISRDNAKNSLYLQMNSLRAEDTAVYYCARLPGFNPPFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

(19) United States
(12) Patent Application Publication
(10) Pub. No.: US 2025/0326835 A1
(43) Pub. Date: Oct. 23, 2025

(54) USE OF ANTI-CLAUDIN-1 ANTIBODIES TO TREAT CHOLANGIOCARCINOMA

(71) Applicants: Alentis Therapeutics AG, Allschwil (CH); Université de Strasbourg, Strasbourg (FR); Institut National de la Santé et de la Recherche Médicale, Paris (FR)

(72) Inventors: Thomas BAUMERT, Strasbourg (FR); Markus MEYER, Allschwil (CH); Roberto IACONE, Allschwil (CH); Alberto TOSO, Allschwil (CH); Tamás SCHWEIGHOFFER, Allschwil (CH); Geoffrey TEIXEIRA, Allschwil (CH); Laurent MAILLY, Ernolsheim-Bruche (FR); Marion MULLER, Strasbourg (FR); Zeina Hussein NEHME, Strasbourg (FR)

(21) Appl. No.: 18/870,222

(22) PCT Filed: Jun. 1, 2023
(86) PCT No.: PCT/IB2023/055667

§ 371 Date: Nov. 27, 2024

Related U.S. Application Data
(60) Provisional application No. 63/365,681, filed on Jun. 1, 2022.

(51) Int. Cl. C07K 16/28; A61K 31/7068; A61K 33/243; A61K 39/00; A61P 35/00; A61P 35/04

(52) U.S. Cl. CPC C07K 16/28; A61K 31/7068; A61K 33/243; A61P 35/00; A61P 35/04; A61K 2039/505; A61K 2039/54; C07K 2317/24; C07K 2317/565

(57) ABSTRACT

The present disclosure relates to a method of treating a cholangiocarcinoma in a human subject in need thereof, comprising administering a therapeutically effective amount of an anti-Claudin-1 antibody to the human subject.

### CROSS REFERENCE TO RELATED APPLICATIONS

This application claims the priority benefit of U.S. Provisional App. No. 63/365,681, filed Jun. 1, 2022, which is hereby incorporated by reference in its entirety.

### REFERENCE TO SEQUENCE LISTING SUBMITTED ELECTRONICALLY VIA EFS-WEB

The content of the electronically submitted sequence listing (Name: 4872_020PC01_SequenceListing_ST26; Size: 20,121 bytes; and Date of Creation: May 29, 2023), filed with the application, is incorporated herein by reference in its entirety.

### FIELD OF THE INVENTION

According to various aspects of this disclosure, the present disclosure relates to methods of treating cholangiocarcinoma (CCA).

### BACKGROUND OF THE INVENTION

Cholangiocarcinoma (CCA) is a highly aggressive adenocarcinoma of the hepatobiliary system (Brindley et al., 7 Nat Rev Dis Primers 1-17 (2021)). Arising along the biliary tree and/or within the hepatic parenchyma, CCA is considered a heterogeneous malignancy that is mainly divided into three primary anatomic subtypes, namely intrahepatic CCA (iCCA), perihilar CCA (pCCA) or distal CCA (dCCA) (Banales et al., 17 Nat Rev Gastroenterol Hepatol 557-588 (2020)). Ranking after hepatocellular carcinoma (HCC) as the second most common primary hepatic malignancy, CCA embodies approximately 15% of all primary liver tumors and 2% of cancer-related deaths worldwide (Vaquero et al., 13 Nat Rev Gastroenterol Hepatol 261-280 (2016)). Although considered a rare cancer, CCA incidence rates have raised dramatically in the last decade (Bertuccio et al., 71(1) J Hepatol. 104-114 (July 2019)), ranging from 1 to more than 4 cases/100,000 in European countries (Al Mahjoub et al., 31(6) Eur J Gastroenterol Hepatol 678-684 (June 2019)). This could be partly explained by an increasing trend of CCA risk factors globally, for instance diabetes, primary sclerosing cholangitis, hepatolithiasis, cirrhosis, hepatitis B and hepatitis C (Clements et al., 72(1) J Hepatol. 95-103 (January 2020)). In particular, chronic biliary inflammation and/or cholestasis are identified as major drives of CCA initiation, highlighting the prominent role played by the tumor microenvironment (TME), notably cancer-associated fibroblasts (CAFs), tumor-associated macrophages (TAMs), pro-inflammatory cytokines and growth factors (Leyva-Illades et al., 1(1) Transl Gastrointest Cancer 71-80 (2012)). Those extracellular players intersect with the deregulation of some intracellular signal transduction pathways considered as key drivers of cholangiocarcinogenesis, such as receptor tyrosine kinase (RTK) signaling, RAS-RAF-ERK, PI3K-AKT-mTOR, Notch, Hedgehog, and Wnt signaling (Yang J et al., 21(5) Expert Opin Ther Targets 485-498 (May 2017)).

CCA is usually diagnosed very late, resulting in aggressive disease progression, poor treatment response, and dismal prognosis with a median survival of less than 2 years (Vaquero et al., 13 Nat Rev Gastroenterol Hepatol 261-280 (2016)). Although curative hepatic resection is a therapeutic option for CCA management, only 25% of patients are eligible due to metastatic or locally advanced tumors, with 50% of operated patients achieving curative or margin-free resection (Nagorney et al., 40 Adv Surg 159-171 (2006)). First-line standard-of-care chemotherapies include gemcitabine and cisplatin combination, in addition to the recently approved targeted therapies, e.g., Pemigatinib, a fibroblast growth factor receptor inhibitor and Ivosidenib, an isocitrate dehydrogenase 1 (IDH1) mutant inhibitor, that can improve patient's outcome, but results in response in only a very small subset of advanced stage and/or metastatic CCA, with important side effects and potential therapeutic resistance (Sasaki et al., 10(14) J Clin Med 3108 (2021)).

A growing body of evidence is pointing toward expression deregulation of members of the claudin family in CCA and their contribution to carcinogenesis (Németh et al., 57(2) J Histochem Cytochem 113-121 (February 2009)). Claudin-1 (CLDN1) is a transmembrane protein expressed in tight junctions (TJs), but also in a non-junctional form, e.g., at the basolateral membrane of the human hepatocyte, where it serves as a cell entry factor of hepatitis C virus (Evans et al., 446 Nature 801-805 (2007)). CLDN1 was previously identified as a mediator and therapeutic target for liver fibrosis and hepatocellular carcinoma (HCC) (WO 20161/46809 A1). Extensive studies in non-human primates and mouse models did not reveal any major toxicity even when high doses of the mAb largely exceeding the therapeutic need were repeatedly applied.

Given the absence of efficient drugs combined with the rising incidence of the disease, there is an urgent unmet medical need for novel first-line therapeutic approaches to treat CCA and improve patient outcomes.

### BRIEF SUMMARY OF THE INVENTION

The present disclosure provides a method of treating a cholangiocarcinoma (CCA) in a human subject in need thereof, comprising administering a therapeutically effective amount of an anti-Claudin-1 antibody to the human subject.

In some aspects, provided herein is an anti-Claudin-1 antibody or a pharmaceutical composition thereof for use in a method of treating a cholangiocarcinoma (CCA) in a human subject, the method comprising administering an effective amount of the anti-Claudin-1 antibody or a pharmaceutical composition thereof to the human subject.

In some aspects, provided herein is a kit for treating a subject suffering from a cholangiocarcinoma, comprising a therapeutically effective amount of an anti-Claudin-1 antibody and an insert comprising instructions for use of the kit.

In some aspects, provided herein is a pharmaceutical composition for the treatment of a cholangiocarcinoma, comprising a therapeutically effective amount of an anti-Claudin-1 antibody.

In some aspects, Claudin-1 (CLDN1) is overexpressed in the human subject compared to expression levels in a normal subject.

In some aspects, the human subject is further administered a chemotherapy. In some aspects, the chemotherapy is gemcitabine. In some aspects, the chemotherapy is cisplatin.

In some aspects, the anti-Claudin-1 antibody comprises the six complementary determining regions (CDRs) of an anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody comprises a heavy chain variable domain complementary determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7, and/or a light chain variable domain complementary determining region (CDR) L1 comprising the amino acid sequence set forth in SEQ ID NO: 8, a CDR L2 comprising the amino acid sequence GAS, and a CDR L3 comprising the amino acid sequence set forth in SEQ ID NO: 10.

In some aspects, the anti-Claudin-1 antibody is humanized.

In some aspects, the anti-Claudin-1 antibody comprises a VH comprising the amino acid sequence set forth in SEQ ID NO: 3 or SEQ ID NO: 13.

In some aspects, the anti-Claudin-1 antibody comprises a VL comprising the amino acid sequence set forth in SEQ ID NO: 4 or SEQ ID NO: 14.

In some aspects, the anti-Claudin-1 antibody comprises a VH comprising the amino acid sequence set forth in SEQ ID NO: 3; and a VL comprising the amino acid sequence set forth in SEQ ID NO: 4.

In some aspects, the anti-Claudin-1 antibody comprises a VH comprising the amino acid sequence set forth in SEQ ID NO: 13; and a VL comprising the amino acid sequence set forth in SEQ ID NO: 14.

In some aspects, the anti-Claudin-1 antibody is administered intratumorally, intravenously, intraperitoneally, intramuscularly, intrathecally or subcutaneously.

In some aspects, the CCA is an intrahepatic CCA.

In some aspects, the CCA is a perihilar CCA.

In some aspects, the CCA is a distal CCA

In some aspects, the CCA is a combined or mixed hepatocellular cholangiocarcinoma (cHCC-CCA).

In some aspects, the CCA is metastatic.

In some aspects, the CCA is treated with chemotherapy such as gemcitabine and cisplatin.

In some aspects, the CCA contains the following genetic mutations: Isocitrate Dehydrogenase (NADP(+)) 1 (IDH1), Isocitrate Dehydrogenase (NADP(+)) 2 (IDH2), BRCA1 Associated Protein 1 (BAP1), Fibroblast Growth Factor Receptor 2 (FGFR2), Kirsten Rat Sarcoma Viral Oncogene Homologue (KRAS), Polybromo 1 (PBRM1), AT-Rich Interaction Domain 1A (ARID1A), Phosphatidylinositol-4,5-Bisphosphate 3-Kinase Catalytic Subunit Alpha (PIK3CA), Ephrin type-A receptor 2 (EPHA2), Cyclin-Dependent Kinase Inhibitor 2A (CDKN2A), Tumor Protein P53 (TP53), SMAD Family Member 4 (SMAD4), Transforming Growth Factor Beta Receptor 2 (TGFBR2).

In some aspects, the method or use provided herein further comprises administering a chemotherapeutic drug to the human subject in need thereof.

In some aspects, the chemotherapeutic agent is gemcitabine.

In some aspects, the chemotherapeutic agent is cisplatin.

### DESCRIPTION OF FIGURES

FIGS. 1A-1C (FIGS. 1A-1C) show transcriptional profiling using bulk RNA-sequencing (RNA-seq) datasets deposited in the Genomic Data Commons Data portal, such as extrahepatic CLDN1 expression in comparison to non-tumoral bile duct samples—GSE13205 (FIG. 1A), CLDN1 expression in proliferative intrahepatic CCA in comparison to non-tumoral bile duct samples—GSE32225 (FIG. 1), and CLDN1 expression in proliferative CCA in comparison to inflammatory CCA—GSE32225 (FIG. 1C).

FIG. 1D (FIG. 1D) shows CLDN1 expression in patient CCA tumor tissues with different genetic driver mutations based on bulk RNA-sequencing (RNA-seq) datasets deposited in the Genomic Data Commons Data portal—GSE89747 and GSE89748. The respective CCA mutations are indicated on the x-axis and CLDN1 expression (signal intensity) is shown on the y-axis.

FIG. 2A (FIG. 2A) shows the experimental approach of an exemplary model for a cell line-derived xenograft (CDX) NRG mouse model.

FIGS. 2B-2D (FIGS. 2B-2D) show tumor volume (FIG. 2B) and grade of skin ulceration (FIG. 2C-2D) in EGI-1 CDX models after treatment with either an anti-CLDN1 H3L3 antibody or control.

FIGS. 2E-2F (FIGS. 2E-2F) show tumor volume (FIGS. 2E-2F) in HuCC-A1 CDX models after treatment with either an anti-CLDN1 H3L3 antibody or control.

FIG. 2G (FIG. 2G) shows tumor volume in HuCC-T1 CDX models after treatment with either an anti-CLDN1 H3L3 antibody or control.

FIG. 2H (FIG. 2H) shows tumor volume in a PDX mouse model after treatment with either an anti-CLDN1 H3L3 antibody or control.

FIGS. 3A-3C (FIGS. 3A-3C) show the effect of anti-CLDN1 H3L3 antibodies on cell migration in a wound healing assay of an intrahepatic CCA cell line (HuCC-A1) and an extrahepatic CCA cell line (KKU100). FIG. 3A shows representative photomicrographs of the cell monolayer of HuCC-A1 or KKU100 cells co-cultured with LX2 cells 24 h after wounding. FIG. 3B shows the effect of treatment of anti-CLDN1 H3L3 and control antibodies on wound closure assay in the HuCC-A1 assay cells after 24 hours. FIG. 3C shows the effect of treatment of CLDN1-specific (H3L3) and control antibodies on a wound closure assay in the KKU100 cells after 24 hours (Full wound closure=100%). Scale bars indicate 330 μm.

FIGS. 3D-3G (FIGS. 3D-3G) show the effect of the anti-CLDN1 H3L3 antibodies on cell invasion of an extrahepatic (EGI-1) and intrahepatic (HuCC-T1) CCA cell lines using a transwell cancer invasion assay. FIG. 3D shows the experimental approach of the transwell Matrigel invasion assay. FIG. 3E shows representative images of crystal violet staining visualizing invading EGI-1 or HuCC-T1 cells in transwell chamber assays. FIG. 3F shows the effect of treatment of anti-CLDN1 H3L3 and control antibodies on Matrigel invasion in the EGI-1 cells transwell assay. FIG. 3G shows the effect of anti-CLDN1 H3L3 and control antibody treatment on Matrigel invasion in a HuCC-T1 cells transwell assay. Scale bars indicate 330 m.

FIG. 4A (FIG. 4A) shows RNA-seq analysis comparing the effect of treatment with anti-CLDN1 H3L3 antibody v. control on HuCC-A1 tumor tissue from CDX mice.

FIG. 4B (FIG. 4B) shows Western Blot analyses of key signaling pathways in EGI-1 CDX tumor samples after treatment with anti-CLDN1 H3L3 antibody v. control treated animals.

FIG. 4C (FIG. 4C) shows a quantification analysis of the Western Blot in FIG. 4B.

FIGS. 4D-4F (FIGS. 4D-4F) show the effect of treatment of anti-CLDN1 H3L3 v. isotype control antibodies on Notch1 (FIG. 4D), SRC (FIG. 4E), and FAK signaling (FIG. 4F) in EGI-1/LX2 and EGI-1 cells using Western Blot analyses. Proteins and molecular weight are indicated.

FIG. 4G (FIG. 4G) shows an RNA-Seq analysis in HuCC-A1 tumor tissue derived from CDX model shown in FIG. 2A. Comparative analyses of tumor tissues of mice treated with anti-CLDN1 H3L3 antibody v. control are shown demonstrating differential expression of downstream targets of Notch1, SRC and FAK signaling pathways shown in FIGS. 4D-4F.

FIG. 5A (FIG. 5A) shows the experimental approach of a CCA metastasis model using intravenous retro-orbital HuCC-T1 cells injection with subsequent analyses of lung metastasis in anti-CLDN1 H3L3 mAb v. control-treated mice.

FIGS. 5B-5C (FIGS. 5B-5C) show immunohistochemistry and quantification of CCA HuCC-T1 cell metastases in lung sections visualized and detected by CK18 staining in mice treated with either anti-CLDN1 H3L3 antibody or control.

FIG. 6A (FIG. 6A) shows flow cytometry analyses of CLDN1 expression using CLDN1-specific H3L3 mAb in EGI-1 cells after treatment with cisplatin and gemcitabine.

FIG. 6B (FIG. 6B) shows the delta-mean fluorescence intensity (AMFI) fold change of the flow cytometry analyses corresponding to CLDN1 expression detected by CLDN1-specific mAb H3L3 shown in FIG. 6A.

FIG. 6C (FIG. 6C) shows Western Blot analyses of CLDN1 expression in EGI-1 cholangiocarcinoma cells after treatment with cisplatin and gemcitabine.

FIG. 6D (FIG. 6D) shows a quantification analysis of the proteins shown in Western Blots of FIG. 6C.

FIG. 7A-7B (FIGS. 7A-7B) show body weight (FIG. 7A) and tumor volume (FIG. 7B) in anti-CLDN1 H1L1 mAb v. control-treated mice.

### DETAILED DESCRIPTION OF THE INVENTION

### I. Definitions

Unless defined otherwise, all technical and scientific terms used herein have the same meaning as commonly understood by one of ordinary skill in the art to which this disclosure belongs. In case of conflict, the present application including the definitions will control. Unless otherwise required by context, singular terms shall include pluralities and plural terms shall include the singular. All publications, patents and other references mentioned herein are incorporated by reference in their entireties for all purposes as if each individual publication or patent application were specifically and individually indicated to be incorporated by reference.

Although methods and materials similar or equivalent to those described herein can be used in practice or testing of the present disclosure, suitable methods and materials are described below. The materials, methods and examples are illustrative only and are not intended to be limiting. Other features and advantages of the disclosure will be apparent from the detailed description and from the claims.

In order to further define this disclosure, the following terms and definitions are provided.

The singular forms “a,” “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

The term “about” is used herein to mean approximately, roughly, around, or in the regions of. When the term “about” is used in conjunction with a numerical range, it modifies that range by extending the boundaries above and below the numerical values set forth. In general, the term “about” is used herein to modify a numerical value above and below the stated value by a variance of 10 percent, up or down (higher or lower).

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6, etc., as well as individual numbers within that range, for example, 1, 2, 3, 4, 5, and 6. This applies regardless of the breadth of the range. Numeric ranges recited are inclusive of the numbers defining the range and include each integer within the defined range.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10.

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “treating” or “treatment” as used herein refers to the administration of a composition to a subject for therapeutic purposes.

The term “human Claudin-1 (or CLDN1)” refers to a protein having the sequence shown in NCBI Accession Number NP_066924.1, or any naturally occurring variants commonly found in HCV permissive human populations.

The term “antibody”, as used herein, refers to any immunoglobulin that contains an antigen binding site that immunospecifically binds an antigen. As such, the term antibody encompasses not only whole antibody molecules, but also antibody fragments as well as variants (including derivatives) of antibodies and of antibody fragments as long as the derivatives and fragments maintain specific binding ability. The term encompasses monoclonal antibodies and polyclonal antibodies. The term also covers any protein having a binding domain, which is homologous or largely homologous to an immunoglobulin-binding domain. These proteins may be derived from natural sources, or partly or wholly synthetically produced. The term “specific binding”, when used in reference to an antibody, refers to an antibody binding to a predetermined antigen. Typically, the antibody binds with an affinity of at least 1×10⁷ M¹, and binds to the predetermined antigen with an affinity that is at least two-fold greater than the affinity for binding to a non-specific antigen (e.g., BSA, casein).

As used herein, the term “humanized antibody” refers to a chimeric antibody comprising amino acid residues from non-human hypervariable regions and amino acid residues from human framework regions (FRs). In particular, a humanized antibody comprises all or substantially all of at least one, typically two, variable domains, in which all or substantially all of the complementarity determining regions (CDRs) are those of a human antibody. A humanized antibody optionally may comprise at least a portion of an antibody constant region derived from a human antibody. A “humanized form” of an antibody, e.g., a non-human antibody, refers to an antibody that has undergone humanization.

It is understood that wherever aspects are described herein with the language “comprising,” otherwise analogous aspects described in terms of “consisting of” and/or “consisting essentially of” are also provided.

As used herein, the term “administering” refers to the physical introduction of a composition comprising a therapeutic agent (e.g., an anti-Claudin-1 antibody) to a subject, using any of the various methods and delivery systems known to those skilled in the art. Routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include a topical, epidermal or mucosal route of administration, for example, intranasally, vaginally, rectally, sublingually or topically. Administering can also be performed, for example, once, a plurality of times, and/or over one or more extended periods.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to prevent or delay other unwanted cell proliferation. In some aspects, an effective amount is an amount sufficient to delay tumor development. In some aspects, an effective amount is an amount sufficient to prevent or delay tumor recurrence. An effective amount can be administered in one or more administrations. The effective amount of the drug or composition may: (i) reduce the number of cancer cells; (ii) reduce tumor size; (iii) inhibit, retard, slow to some extent and may stop cancer cell infiltration into peripheral organs; (iv) inhibit (i.e., slow to some extent and may stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of anti-Claudin-1 antibody clinically proven to affect a significant decrease in cancer or slowing of progression of cancer, such as an advanced solid tumor.

A “patient” as used herein includes any patient who is afflicted with a cancer (e.g., a fibrotic cancer). The terms “subject” and “patient” are used interchangeably herein.

### II. Anti-Claudin-1 Antibodies

The present invention concerns the use of anti-Claudin-1 antibodies for the treatment of a cholangiocarcinoma in a human subject in need thereof. In some aspects, disclosed herein is a method of treating a cholangiocarcinoma in a human subject in need thereof comprising administering a therapeutically effective amount of an anti-Claudin-1 antibody to the human subject.

CLDN1 is a transmembrane protein with two major roles: (1) together with other proteins it contributes to the barrier function by tight junctions; (2) it is expressed outside the tight junctions in the basolateral membrane of epithelial cells, where CLDN1 has been shown to mediate procarcinogenic signaling, epithelial-mesenchymal transition (EMT) and cell fate. Moreover, it has been shown that CLDN1 is also expressed by nonepithelial cells such as myofibroblasts of liver, lung, and kidney.

Antibodies directed against human Claudin-1 have been previously described to treat hepatitis c virus infection, hepatocellular carcinoma, and certain fibrotic diseases, such as lung fibrosis (see WO 2010/034812, WO 2016/146809, and WO 2021/094469). Anti-Claudin-1 antibodies that can be used in the practice of the present invention include any antibody raised against Claudin-1. Examples are disclosed in WO 2010/034812 and WO 2017/162678.

Other examples of suitable anti-Claudin-1 antibodies include those disclosed in European Patent No. EP 1 167 389, in U.S. Pat. No. 6,627,439, in international patent application published under No. WO 2014/132307, in international patent applications published under No. WO 2015/014659 and No. WO 2015/014357, and in Yamashita et al., 353(1) J. Pharmacol. Exp. Ther. 112-118 (2015).

Anti-Claudin-1 antibodies suitable for use in the present invention may be polyclonal antibodies or monoclonal antibodies.

Anti-Claudin-1 antibodies suitable for use according to the present invention may also be “humanized”: sequence differences between rodent antibodies and human sequences can be minimized by replacing residues which differ from those in the human sequences by site-directed mutagenesis of individual residues or by grafting of entire regions or by chemical synthesis. Humanized antibodies can also be produced using recombinant methods. In the humanized form of the antibody, some, most or all of the amino acids outside the CDR regions are replaced with amino acids from human immunoglobulin molecules, while some, most or all amino acids within one or more CDR regions are unchanged. Small additions, deletions, insertions, substitutions or modifications of amino acids are permissible as long as they do not significantly modify the biological activity of the resulting antibody. Suitable human “replacement” immunoglobulin molecules include IgG1, IgG2, IgG2a, IgG2b, IgG3, IgG4, IgA, IgM, IgD or IgE molecules, and fragments thereof.

In some aspects, a humanized anti-Claudin-1 antibody for use according to the present invention is one previously described in WO 2017/162678. Exemplary sequences for the antibody or antigen binding fragment provided herein are described in Table 1.

TABLE 1
Exemplary Sequences

| Description | Sequence | SEQ ID NO: |
|---|---|---|
| H1L1- | EVQLVESGGGLVKPGGSLRLSCAASGFSFSSYGMNWVRQA | 1 |
| Heavy | PGKGLEWVSSISPSGSYFYYADSVKGRFTISRDNAKNSLYLQ | |
| Chain #1 | MNSLRAEDTAVYYCARLPGFNPPFDHWGQGTLVTVSSA | |
| (ALE.F02) | STKGPSVFPLAPSSKSTSGGTAALGCLVKDYFPEPVTVSWNS | |
| | GALTSGVHTFPAVLQSSGLYSLSSVVTVPSSSLGTQTYICNV | |
| | NHKPSNTKVDKRVEPKSCDKTHTCPPCPAPEFEGGP | |
| | SVFLFPPKPKDTLMISRTPEVTCVVVDVSHEDPEVKFNWYV | |
| | DGVEVHNAKTKPREEQYNSTYRVVSVLTVLHQDWLNGKE | |
| | YKCKVSNKALPASIEKTISKAKGQPREPQVYTLPPSREEM | |
| | TKNQVSLTCLVKGFYPSDIAVEWESNGQPENNYKTTPPVLD | |
| | SDGSFFLYSKLTVDKSRWQQGNVFSCSVMHEALHNHYTQK | |
| | SLSLSPG | |
| H1L1- | DIQMTQSPATLSVSPGERATLSCKASQNVGGNVDWYQWKP | 2 |
| Light Chain | GQAPRLLIYGASNRYTGIPARFRGSGSGTEFTLTISSLQSEDF | |
| | AVYYCLQYKNNPWTFGQGTKVEIKRTVAAPSVFIFPPSDEQ | |
| | LKSGTASVVCLLNNFYPREAKVQWKVDNALQSGNSQESVT | |
| | EQDSKDSTYSLSSTLTLSKADYEKHKVYACEVTHQGLSSPV | |
| | TKSFNRGEC | |
| H1L1-VH | EVQLVESGGGLVKPGGSLRLSCAASGFSFSSYGMNWVRQA | 3 |
| | PGKGLEWVSSISPSGSYFYYADSVKGRFTISRDNAKNSLYLQ | |
| | MNSLRAEDTAVYYCARLPGFNPPFDHWGQGTLVTVSS | |
| H1L1-VL | DIQMTQSPATLSVSPGERATLSCKASQNVGGNVDWYQWKP | 4 |
| | GQAPRLLIYGASNRYTGIPARFRGSGSGTEFTLTISSLQSEDF | |
| | AVYYCLQYKNNPWTFGQGTKVEIK | |
| H1L1/H3L3- | GFSFSSYG | 5 |
| CDR H1 | | |
| H1L1/H3L3- | ISPSGSYF | 6 |
| CDR H2 | | |
| H1L1/H3L3- | PGFNPPFDH | 7 |
| CDR H3 | | |
| H1L1/H3L3- | QNVGGN | 8 |
| CDR L1 | | |
| H1L1/H3L3- | GAS | - |
| CDR L2 | | |
| H1L1/H3L3- | LQYKNNPWT | 10 |
| CDR L3 | | |
| H3L3- | QVQLVESGGGVVQPGRSLRLSCLGSGFSFSSYGMNWVRQA | 11 |
| Heavy | PGKGLEWVASISPSGSYFYYADSVKGRFTISRDNSKNTLYLQ | |
| Chain | MTSLRAEDTAIYYCARLPGFNPPFDHWGQGTLVTVSSASTK | |
| | GPSVFPLAPSSKSTSGGTAALGCLVKDYFPEPVTVSWNSGA | |
| | LTSGVHTFPAVLQSSGLYSLSSVVTVPSSSLGTQTYICNVNH | |
| | KPSNTKVDKRVEPKSCDKTHTCPPCPAPELLGGPSVFLFPPK | |
| | PKDTLMISRTPEVTCVVVDVSHEDPEVKFNWYVDGVEVHN | |
| | AKTKPREEQYNSTYRVVSVLTVLHQDWLNGKEYKCKVSN | |
| | KALPAPIEKTISKAKGQPREPQVYTLPPSREEMTKNQVSLTC | |
| | LVKGFYPSDIAVEWESNGQPENNYKTTPPVLDSDGSFFLYS | |
| | KLTVDKSRWQQGNVFSCSVMHEALHNHYTQKSLSLSPG | |
| H3L3- | DIQMTQSPSSLSASVGDRVTITCKASQNVGGNVDWYQWKP | 12 |
| Light Chain | GKAPKLLIYGASNRYTGVPDRFRGSGSGTDFTLTISSLQPED | |
| | VATYYCLQYKNNPWTFGGGTKVEIKRTVAAPSVFIFPPSDE | |
| | QLKSGTASVVCLLNNFYPREAKVQWKVDNALQSGNSQESV | |
| | TEQDSKDSTYSLSSTLTLSKADYEKHKVYACEVTHQGLSSP | |
| | VTKSFNRGEC | |
| H3L3-VH | QVQLVESGGGVVQPGRSLRLSCLGSGFSFSSYGMNWVRQA | 13 |
| | PGKGLEWVASISPSGSYFYYADSVKGRFTISRDNSKNTLYLQ | |
| | MTSLRAEDTAIYYCARLPGFNPPFDHWGQGTLVTVSS | |
| H3L3-VL | DIQMTQSPSSLSASVGDRVTITCKASQNVGGNVDWYQWKP | 14 |
| | GKAPKLLIYGASNRYTGVPDRFRGSGSGTDFTLTISSLQPED | |
| | VATYYCLQYKNNPWTFGGGTKVEIK | |
| H1L1- | EVQLVESGGGLVKPGGSLRLSCAASGFSFSSYGMNWVRQA | 15 |
| Heavy | PGKGLEWVSSISPSGSYFYYADSVKGRFTISRDNAKNSLYLQ | |
| Chain #2 | MNSLRAEDTAVYYCARLPGFNPPFDHWGQGTLVTVSSAST | |
| (ALE.C04) | KGPSVFPLAPSSKSTSGGTAALGCLVKDYFPEPVTVSWNSG | |
| | ALTSGVHTFPAVLQSSGLYSLSSVVTVPSSSLGTQTYICNVN | |
| | HKPSNTKVDKRVEPKSCDKTHTCPPCPAPELLGGPSVFLFPP | |
| | KPKDTLMISRTPEVTCVVVDVSHEDPEVKFNWYVDGVEVH | |
| | NAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLY | |
| | SKLTVDKSRWQQGNVFSCSVMHEALHNHYTQKSLSLSPG | |

In some aspects, the anti-Claudin-1 antibody comprises a complementarity determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

In some aspects, the anti-Claudin-1 antibody comprises a complementarity determining region (CDR) L1 comprising the amino acid sequence set forth in SEQ ID NO: 8, a CDR L2 comprising the amino acid sequence set forth as GAS, and a CDR L3 comprising the amino acid sequence set forth in SEQ ID NO: 10.

In some aspects, the complementarity determining regions (CDRs) disclosed herein are defined according to IMGT®. However, it is appreciated that other methods of defining the CDRs in the art can also be used.

In some aspects, the six complementarity determining regions (CDRs) of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the heavy chain variable region (“VH”) and the light chain variable region (“VL”) of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody comprises a VH comprising the amino acid sequence set forth in SEQ ID NO: 3 or SEQ ID NO: 13.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 3.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 13.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 3.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 13.

In some aspects, the anti-Claudin-1 antibody comprises a VL comprising the amino acid sequence set forth in SEQ ID NO: 4 or SEQ ID NO: 14.

In some aspects, the anti-Claudin-1 antibody comprises a VL comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 4.

In some aspects, the anti-Claudin-1 antibody comprises a VL comprising an amino acid sequence having at least 85%, at least 90%, at least 91%, at least 92%, at least 93%, at least 94%, at least 95%, at least 96%, at least 97%, at least 98%, at least 99% sequence identity to SEQ ID NO: 14.

In some aspects, the anti-Claudin-1 antibody comprises a VL comprising an amino acid sequence having about 85%, about 90%, about 91%, about 92%, about 93%, about 94%, about 95%, about 96%, about 97%, about 98%, about 99%, or about 100% sequence identity to SEQ ID NO: 4.

In some aspects, the anti-Claudin-1 antibody comprises a VL comprising an amino acid sequence having about 85%, about 90%, about 91%, about 92%, about 93%, about 94%, about 95%, about 96%, about 97%, about 98%, about 99%, or about 100% sequence identity to SEQ ID NO: 14.

In some aspects, the anti-Claudin-1 antibody comprises a VH comprising the amino acid sequence set forth in SEQ ID NO: 3; and a VL comprising the amino acid sequence set forth in SEQ ID NO: 4.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 3 and a light chain variable region (VL) comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 4.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 3 and a light chain variable region (VL) comprising an amino acid sequence having about 85%, about 90%, about 91%, about 92%, about 93%, about 94%, about 95%, about 96%, about 97%, about 98%, about 99%, or about 100% sequence identity to SEQ ID NO: 4.

In some aspects, the anti-Claudin-1 antibody comprises a VH comprising the amino acid sequence set forth in SEQ ID NO: 13; and a VL comprising the amino acid sequence set forth in SEQ ID NO: 14.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 13 and a light chain variable region (VL) comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 14.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 13 and a light chain variable region (VL) comprising an amino acid sequence having about 85%, about 90%, about 91%, about 92%, about 93%, about 94%, about 95%, about 96%, about 97%, about 98%, about 99%, or about 100% sequence identity to SEQ ID NO: 14.

In some aspects, the heavy chain and light chain of the anti-Claudin-1 antibody are the same as those in the anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody comprises a heavy chain comprising the amino acid sequence set forth in SEQ ID NO: 1 or SEQ ID NO: 15.

In some aspects, the anti-Claudin-1 antibody comprises a heavy chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 1 or SEQ ID NO: 15.

In some aspects, the anti-Claudin-1 antibody comprises a heavy chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 1 or SEQ ID NO: 15.

In some aspects, the anti-Claudin-1 antibody comprises a light chain comprising the amino acid sequence set forth in SEQ ID NO: 2.

In some aspects, the anti-Claudin-1 antibody comprises a light chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 2.

In some aspects, the anti-Claudin-1 antibody comprises a light chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 2.

In some aspects, the anti-Claudin-1 antibody comprises a heavy chain comprising the amino acid sequence set forth in SEQ ID NO: 1 or SEQ ID NO: 15; and a light chain comprising the amino acid sequence set forth in SEQ ID NO: 2.

In some aspects, the anti-Claudin-1 antibody comprises a heavy chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 1 or SEQ ID NO: 15 and a light chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 2.

In some aspects, the anti-Claudin-1 antibody comprises a heavy chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 1 or SEQ ID NO: 15 and a light chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 2.

In some aspects, the anti-Claudin-1 antibody comprises a heavy chain comprising the amino acid sequence set forth in SEQ ID NO: 11.

In some aspects, the anti-Claudin-1 antibody comprises a heavy chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 11.

In some aspects, the anti-Claudin-1 antibody comprises a heavy chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 11.

In some aspects, the anti-Claudin-1 antibody comprises a light chain comprising the amino acid sequence set forth in SEQ ID NO: 12.

In some aspects, the anti-Claudin-1 antibody comprises a light chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 12.

In some aspects, the anti-Claudin-1 antibody comprises a light chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 12.

In some aspects, the anti-Claudin-1 antibody comprises a heavy chain comprising the amino acid sequence set forth in SEQ ID NO: 11; and a light chain comprising the amino acid sequence set forth in SEQ ID NO: 12.

In some aspects, the anti-Claudin-1 antibody comprises a heavy chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 11 and a light chain comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 12.

In some aspects, the anti-Claudin-1 antibody comprises a heavy chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 11 and a light chain comprising an amino acid sequence having about 85%, about 90%, about 91%, about 92%, about 93%, about 94%, about 95%, about 96%, about 97%, about 98%, about 99%, or about 100% sequence identity to SEQ ID NO: 12.

The humanized anti-Claudin-1 antibody may be a full monoclonal antibody having an isotope selected from the group consisting of IgG1, IgG2, IgG3 and IgG4. Alternatively, the humanized anti-Claudin-1 antibody may be a fragment of a monoclonal antibody selected from the group consisting of Fv, Fab, F(ab′)₂, Fab′, dsFv, scFv, sc(Fv)₂ and diabodies.

Anti-Claudin-1 antibodies (or biologically active variants or fragments thereof) suitable for use according to the present invention may be functionally linked (e.g., by chemical coupling, genetic fusion, non-covalent association or otherwise) to one or more other molecular entities. Methods for the preparation of such modified antibodies (or conjugated antibodies) are known in the art (see, for example, “Affinity Techniques. Enzyme Purification: Part B”, Methods in Enzymol., 1974, Vol. 34, Jakoby and Wilneck (Eds.), Academic Press: New York, NY; and Wilchek and Bayer, Anal. Biochem., 1988, 171: 1-32). Preferably, molecular entities are attached at positions on the antibody molecule that do not interfere with the binding properties of the resulting conjugate, e.g., positions that do not participate in the specific binding of the antibody to its target.

In some aspects, the anti-Claudin-1 antibodies described herein target the extracellular loop 1 of exposed Claudin-1 outside of tight junctions in the basolateral membrane of epithelial cells.

The antibody molecule and molecular entity may be covalently, directly linked to each other. Or, alternatively, the antibody molecule and molecular entity may be covalently linked to each other through a linker group. This can be accomplished by using any of a wide variety of stable bifunctional agents well known in the art, including homofunctional and heterofunctional linkers.

In some aspects, an anti-Claudin-1 antibody (or a biologically active fragment thereof) for use according to the present invention is conjugated to a detectable agent. Any of a wide variety of detectable agents can be used, including, without limitation, various ligands, radionuclides (e.g., ³H, ¹²⁵I, ¹³¹I, and the like), fluorescent dyes (e.g., fluorescein isothiocyanate, rhodamine, phycoerytherin, phycocyanin, allophycocyanin, o-phthalaldehyde and fluorescamine), chemiluminescent agents (e.g., luciferin, luciferase and aequorin), microparticles (such as, for example, quantum dots, nanocrystals, phosphors and the like), enzymes (such as, for example, those used in an ELISA, i.e., horseradish peroxidase, beta-galactosidase, luciferase, alkaline phosphatase), colorimetric labels, magnetic labels, and biotin, dioxigenin or other haptens and proteins for which antisera or monoclonal antibodies are available.

Other molecular entities that can be conjugated to an anti-Claudin-1 antibody of the present invention (or a biologically active fragment thereof) include, but are not limited to, linear or branched hydrophilic polymeric groups, fatty acid groups, or fatty ester groups.

Thus, in the practice of the present invention, anti-Claudin-1 antibodies can be used under the form of full length antibodies, biologically active variants or fragments thereof, chimeric antibodies, humanized antibodies, and antibody-derived molecules comprising at least one complementarity determining region (CDR) from either a heavy chain or light chain variable region of an anti-Claudin-1 antibody, including molecules such as Fab fragments, F(ab′)2 fragments, Fd fragments, Fabc fragments, Sc antibodies (single chain antibodies), diabodies, individual antibody light single chains, individual antibody heavy chains, chimeric fusions between antibody chains and other molecules, and antibody conjugates, such as antibodies conjugated to a therapeutic agent or a detectable agent. Preferably, anti-Claudin-1 antibody-related molecules according to the present invention retain the antibody's ability to bind its antigen, in particular the extracellular domain of Claudin-1.

### III. Cholangiocarcinoma

Cholangiocarcinoma (CCA) is a highly lethal, epithelial cell malignancy that occurs anywhere along the biliary tree and/or within the hepatic parenchyma. CCA displays features of cholangiocyte differentiation and may arise from the epithelial cells lining the bile ducts, which are termed cholangiocytes. However, the cancers may also develop from peribiliary glands and hepatocytes, depending on the underlying liver disease and location. CCAs are heterogeneous and are best classified according to the primary, anatomic subtype as intrahepatic CCA (iCCA), perihilar CCA (pCCA) or distal CCA (dCCA). iCCA is located proximally to the second-order bile ducts within the liver parenchyma, pCCA is localized between the second-order bile ducts and the insertion of the cystic duct into the common bile duct, and dCCA is confined to the common bile duct below the cystic duct insertion. The true incidence of pCCA and iCCA is unclear owing to the extensive misclassification of pCCA as iCCA in some national databases. In addition, enhanced diagnostic capabilities have enabled increased clinical distinction between carcinoma of unknown primary and iCCA. These factors have contributed to the reported increase in incidence of iCCA over the past two or three decades. Each of the anatomic subtypes is characterized by unique genetic aberrations, clinical presentations and management options. However, many databases categorize both pCCA and dCCA as extrahepatic CCA. Most CCAs are adenocarcinomas and other histological subtypes, such as adenosquamous carcinoma or clear cell carcinoma, are encountered rarely. These cancers are highly desmoplastic and are enmeshed in dense networks of inflammatory cells and matrix termed the tumor immune microenvironment. The epidemiology of these cancers varies worldwide. Infections with specific trematodes (flatworm parasites, commonly called flukes) are a major cause of CCA in some regions. Fluke-related CCA may have a specific pathogenesis, especially genetic aberrations, but the diagnosis and management are not different from non-fluke-related CCA. In the Western world, most patients with CCA do not have an identifiable risk factor, except for some with primary sclerosing cholangitis (PSC) (see Brindley et al., 7 Nat Rev Dis Primers 1-17 (2021)).

In some aspects, the CCA is a combined or mixed hepatocellular cholangiocarcinoma (cHCC-CCA). cHCC-CCA is a distinct type of primary liver cancer sharing unequivocal phenotypical characteristics of both hepatocellular carcinoma (HCC) and cholangiocarcinoma. cHCC-CCA is a rare and aggressive primary hepatic malignancy with significant histological and biological heterogeneity. It presents with more aggressive behavior and worse survival outcomes than either hepatocellular carcinoma or cholangiocarcinoma. The World Health Organization has classified cHCC-CCA into two main types—the classical type, which is characterized by intermixed areas of typical HCC and CCA and the presence of transition zones with intermediate morphology of both types, and the type with stem cell features, which is less common and further subdivided into typical, intermediate, and cholangiocellular subtype. Certain histopathological criteria have been established for the definitive diagnosis of cHCC-CCA, which require the presence of fully differentiated components of hepatocellular and CCA intimately mixed with concurrent evidence of transition zones comprising cells with intermediate morphology. This distinguishes it from HCC and CCAs found in the same liver lobe, which represent collision tumors. Gene panels have revealed that mutations in genes KRAS, ARID1A, TERT promoter, and TP53 associated with the different clinical phenotypes of cHCC-CCA (see Stravraka et al., 6 J Hepatocell Carcinoma 11-21 (2018)).

In some aspects, the cholangiocarcinoma is treated with chemotherapy such as cisplatin and gemcitabine.

### IV. Methods of Use

Methods of the present invention may be accomplished using an anti-Claudin-1 antibody, or a biologically active fragment thereof, or a pharmaceutical composition comprising such an antibody or fragment (see below). These methods generally comprise administration of an effective amount of an anti-Claudin-1 antibody, or biologically active fragment thereof, or of a pharmaceutical composition thereof, to a subject in need thereof (i.e., a subject having a fibrotic tumor). Administration may be performed using any of the administration methods known to one skilled in the art (see below).

The present disclosure provides a method of treating a cholangiocarcinoma (CCA) in a human subject in need thereof, comprising administering a therapeutically effective amount of an anti-Claudin-1 antibody to the human subject.

In some aspects, provided herein is an anti-Claudin-1 antibody or a pharmaceutical composition thereof for use in a method of treating a cholangiocarcinoma (CCA) in a human subject, the method comprising administering an effective amount of the anti-Claudin-1 antibody or a pharmaceutical composition thereof to the human subject.

In some aspects, the CCA is an intrahepatic CCA.

In some aspects, the CCA is a perihilar CCA.

In some aspects, the CCA is a distal CCA

In some aspects, the CCA is a combined or mixed hepatocellular cholangiocarcinoma (cHCC-CCA).

In some aspects, the CCA has a mutation in Isocitrate Dehydrogenase (NADP(+)) 1 (IDH1), Isocitrate Dehydrogenase (NADP(+)) 2 (IDH2), BRCA1 Associated Protein 1 (BAP1), Fibroblast Growth Factor Receptor 2 (FGFR2), Kirsten Rat Sarcoma Viral Oncogene Homologue (KRAS), Polybromo 1 (PBRM1), AT-Rich Interaction Domain 1A (ARID1A), Phosphatidylinositol-4,5-Bisphosphate 3-Kinase Catalytic Subunit Alpha (PIK3CA), Ephrin type-A receptor 2 (EPHA2), Cyclin-Dependent Kinase Inhibitor 2A (CDKNT2A), Tumor Protein P53 (TP53), SMAD Family Member 4 (SMAD4), Transforming Growth Factor Beta Receptor 2 (TGFBR2).

In some aspects, Claudin-1 (CLDN1) is overexpressed in the human subject compared to expression levels in a normal subject.

In some aspects, Claudin-1 (CLDN1) is overexpressed in the human subject treated with chemotherapy.

In some aspects, the anti-Claudin-1 antibody comprises the six complementary determining regions (CDRs) of an anti-Claudin-1 monoclonal antibody secreted by a hybridoma cell line deposited at the DSMZ on Jul. 29, 2008 under an Accession Number DSM ACC2938.

In some aspects, the anti-Claudin-1 antibody is humanized.

In some aspects, the anti-Claudin-1 antibody comprises a VH comprising the amino acid sequence set forth in SEQ ID NO: 3 or SEQ ID NO: 13.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 3.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 13.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 3.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 13.

In some aspects, the anti-Claudin-1 antibody comprises a VL comprising the amino acid sequence set forth in SEQ ID NO: 4 or SEQ ID NO: 14.

In some aspects, the anti-Claudin-1 antibody comprises a VL comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 4.

In some aspects, the anti-Claudin-1 antibody comprises a VL comprising an amino acid sequence having at least 85%, at least 90%, at least 91%, at least 92%, at least 93%, at least 94%, at least 95%, at least 96%, at least 97%, at least 98%, at least 99% sequence identity to SEQ ID NO: 14.

In some aspects, the anti-Claudin-1 antibody comprises a VL comprising an amino acid sequence having about 85%, about 90%, about 91%, about 92%, about 93%, about 94%, about 95%, about 96%, about 97%, about 98%, about 99%, or about 100% sequence identity to SEQ ID NO: 4.

In some aspects, the anti-Claudin-1 antibody comprises a VL comprising an amino acid sequence having about 85%, about 90%, about 91%, about 92%, about 93%, about 94%, about 95%, about 96%, about 97%, about 98%, about 99%, or about 100% sequence identity to SEQ ID NO: 14.

In some aspects, the anti-Claudin-1 antibody comprises a VH comprising the amino acid sequence set forth in SEQ ID NO: 3; and a VL comprising the amino acid sequence set forth in SEQ ID NO: 4.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 3 and a light chain variable region (VL) comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 4.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 3 and a light chain variable region (VL) comprising an amino acid sequence having about 85%, about 90%, about 91%, about 92%, about 93%, about 94%, about 95%, about 96%, about 97%, about 98%, about 99%, or about 100% sequence identity to SEQ ID NO: 4.

In some aspects, the anti-Claudin-1 antibody comprises a VH comprising the amino acid sequence set forth in SEQ ID NO: 13; and a VL comprising the amino acid sequence set forth in SEQ ID NO: 14.

In some aspects, the anti-Claudin-1 antibody comprises a VH comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 13 and a light chain variable region (VL) comprising an amino acid sequence having at least 85%, at least 90%, at least 91%, at least 92%, at least 93%, at least 94%, at least 95%, at least 96%, at least 97%, at least 98%, or at least 99% sequence identity to SEQ ID NO: 14.

In some aspects, the anti-Claudin-1 antibody comprises a VH comprising an amino acid sequence having about 85%, about 90%, about 91%, about 92%, about 93%, about 94%, about 95%, about 96%, about 97%, about 98%, about 99%, or about 100% sequence identity to SEQ ID NO: 13 and a light chain variable region (VL) comprising an amino acid sequence having about 85%, about 90%, about 91%, about 92%, about 93%, about 94%, about 95%, about 96%, about 97%, about 98%, about 99%, or about 100% sequence identity to SEQ ID NO: 14.

In some aspects, the anti-Claudin-1 antibody comprises a complementary determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7.

In some aspects, the anti-Claudin-1 antibody comprises a complementary determining region (CDR) L1 comprising the amino acid sequence set forth in SEQ ID NO: 8, a CDR L2 comprising the amino acid sequence set forth as GAS, and a CDR L3 comprising the amino acid sequence set forth in SEQ ID NO: 10.

In some aspects, the anti-Claudin-1 antibody is administered intratumorally, intravenously, intraperitoneally, intramuscularly, intrathecally or subcutaneously.

In some aspects, the methods further comprise administering a chemotherapeutic agent.

In some aspects, the chemotherapeutic agent is cisplatin.

In some aspects, the chemotherapeutic agent is gemcitabine.

### V. Administration

An anti-Claudin-1 antibody, or a biologically active fragment thereof, (optionally after formulation with one or more appropriate pharmaceutically acceptable carriers or excipients), in a desired dosage, can be administered to a subject in need thereof by any suitable route. Various delivery systems are known and can be used to administer antibodies, including tablets, capsules, injectable solutions, encapsulation in liposomes, microparticles, microcapsules, etc. Methods of administration include, but are not limited to, dermal, intradermal, intramuscular, intraperitoneal, intralesional, intravenous, subcutaneous, intranasal, pulmonary, epidural, and oral routes. An anti-Claudin-1 antibody, or a biologically active fragment thereof, or a pharmaceutical composition thereof, may be administered by any convenient or other appropriate route, for example, by infusion or bolus injection, by absorption through epithelial or mucosa linings (e.g., oral mucosa, bronchial mucosa, rectal and intestinal mucosa, etc.). Administration can be systemic or local.

In some aspects, the anti-Claudin-1 antibody is administered intratumorally, intravenously, intraperitoneally, intramuscularly, intrathecally or subcutaneously.

An anti-Claudin-1 antibody, or a biologically active fragment thereof, (optionally after formulation with one or more appropriate pharmaceutically acceptable carriers or excipients), will be administered in a dosage such that the amount delivered is effective for the intended purpose. The route of administration, formulation and dosage administered will depend on the therapeutic effect desired, the severity of the condition to be treated if already present, the presence of any infection, the age, sex, weight, and general health condition of the patient as well as upon the potency, bioavailability, and in vivo half-life of the antibody or composition used, the use (or not) of concomitant therapies, and other clinical factors. These factors are readily determinable by the attending physician in the course of the therapy. Alternatively or additionally, the dosage to be administered can be determined from studies using animal models (e.g., non-human primates or rodents). Adjusting the dose to achieve maximal efficacy based on these or other methods are well known in the art and are within the capabilities of trained physicians. As studies are conducted using anti-Claudin-1 antibodies, further information will emerge regarding the appropriate dosage levels and duration of treatment.

### VI. Pharmaceutical Compositions

As mentioned above, anti-Claudin-1 antibodies (and related molecules) may be administered per se or as a pharmaceutical composition. Accordingly, the present invention provides pharmaceutical compositions comprising an effective amount of an anti-Claudin-1 antibody, or a biologically active fragment thereof, described herein and at least one pharmaceutically acceptable carrier or excipient.

In some aspects, provided herein is a pharmaceutical composition for the treatment of a cholangiocarcinoma (CCA), comprising a therapeutically effective amount of any of the anti-Claudin-1 antibodies disclosed herein.

In some aspects, the CCA is an intrahepatic CCA.

In some aspects, the CCA is a perihilar CCA.

In some aspects, the CCA is a distal CCA.

In some aspects, the CCA is a combined or mixed hepatocellular cholangiocarcinoma (cHCC-CCA).

The pharmaceutical compositions may be administered in any amount and using any route of administration effective for achieving the desired prophylactic and/or therapeutic effect. The optimal pharmaceutical formulation can be varied depending upon the route of administration and desired dosage. Such formulations may influence the physical state, stability, rate of in vivo release, and rate of in vivo clearance of the administered active ingredient.

The pharmaceutical compositions of the present invention may be formulated in dosage unit form for ease of administration and uniformity of dosage. It will be understood, however, that the total daily dosage of the compositions will be decided by the attending physician within the scope of sound medical judgement.

### VII. Kits

In another aspect, the present invention provides a pharmaceutical pack or kit comprising one or more containers (e.g., vials, ampoules, test tubes, flasks or bottles) containing one or more ingredients of an inventive pharmaceutical composition, allowing administration of an anti-Claudin-1 antibody, or a biologically active fragment thereof.

Different ingredients of a pharmaceutical pack or kit may be supplied in a solid (e.g., lyophilized) or liquid form. Each ingredient will generally be suitable as aliquoted in its respective container or provided in a concentrated form. Pharmaceutical packs or kits may include media for the reconstitution of lyophilized ingredients. Individual containers of the kits will preferably be maintained in close confinement for commercial sale.

Optionally associated with the container(s) can be a notice or package insert in the form prescribed by a governmental agency regulating the manufacture, use or sale of pharmaceutical or biological products, which notice reflects approval by the agency of manufacture, use or sale for human administration. The notice of package insert may contain instructions for use of a pharmaceutical composition according to methods of treatment disclosed herein.

An identifier, e.g., a bar code, radio frequency, ID tags, etc., may be present in or on the kit. The identifier can be used, for example, to uniquely identify the kit for purposes of quality control, inventory control, tracking movement between workstations, etc.

In some aspects, provided herein is a kit for treating a subject suffering from a cholangiocarcinoma (CCA), comprising a therapeutically effective amount of any of the anti-Claudin-1 antibodies disclosed herein and an insert comprising instructions for use of the kit.

In some aspects, the CCA is an intrahepatic CCA.

In some aspects, the CCA is a perihilar CCA.

In some aspects, the CCA is a distal CCA.

In some aspects, the CCA is a combined or mixed hepatocellular cholangiocarcinoma (cHCC-CCA).

### EXAMPLES

The following examples are illustrative and do not limit the scope of the claimed aspects.

### Example 1. CLDN1 is Highly Expressed in Intrahepatic and Extrahepatic CCA Tumors and Correlates with Tumor Stemness

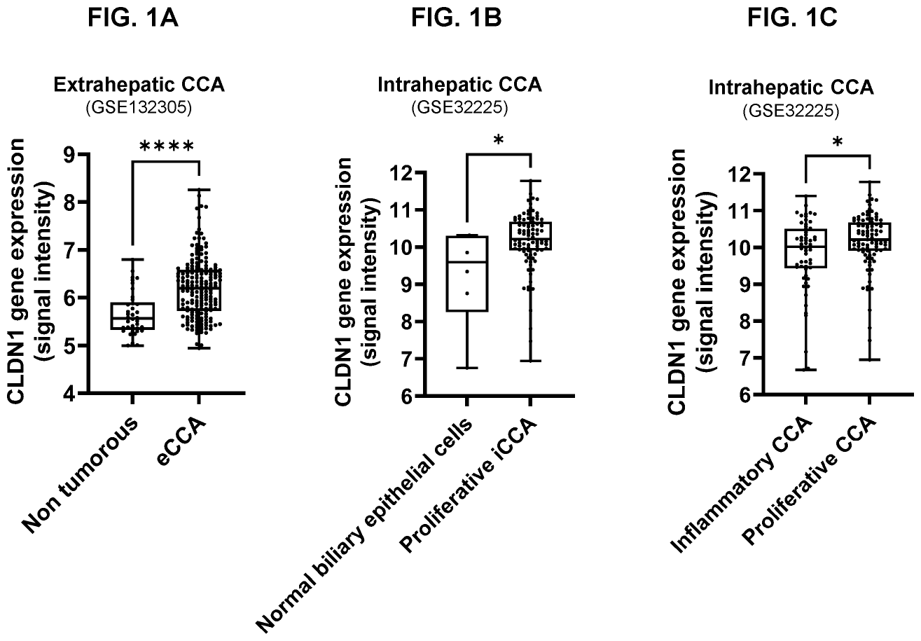

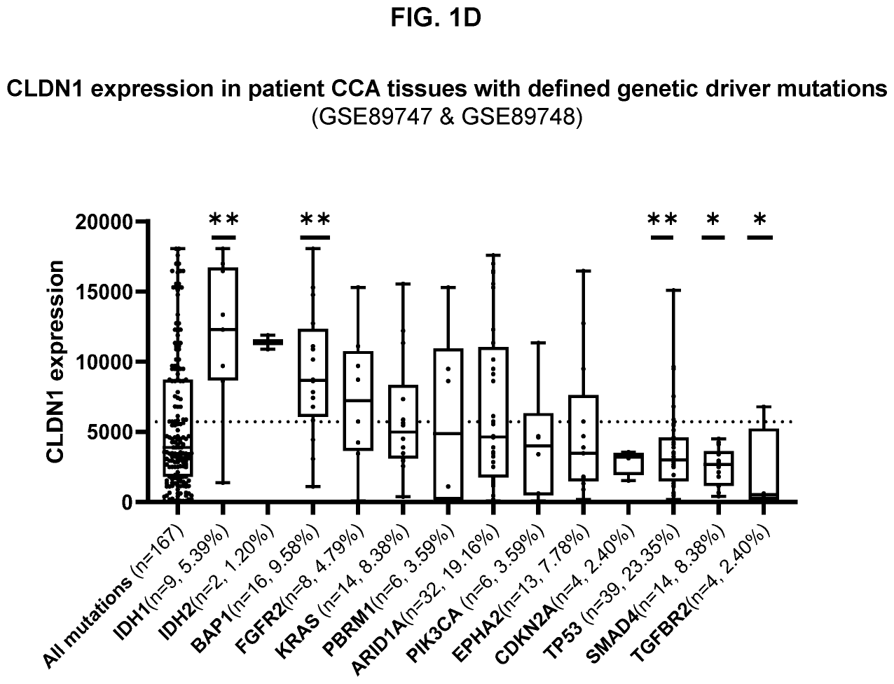

To investigate the role of CLDN1 in bile duct cancer, CLDN1 expression was first analyzed at the transcriptional levels in CCA patients. Comprehensive computational analysis of data retrieved from Genomic Data Commons Data Portal (portal.gdc.cancer.gov) revealed that CLDN1 is highly expressed in extrahepatic (GSE132305, p<0.0001, t-test, FIG. 1A) and proliferative intrahepatic CCA (GSE32225, p=0.03, Mann-Whitney test, FIG. 1B) in comparison to non-tumoral bile duct samples. Interestingly, CLDN1 expression was significantly higher in proliferative compared to inflammatory iCCA (GSE32225, p=0.02, t-test, FIG. 1C).

To investigate the correlation between CLDN1 expression and common mutations in CCA patients, CLDN1 expression was analyzed at the transcriptional level across different common genetic mutations described in CCA patient tumors using a public database (Jusakul et al., 7(10) Cancer Discov 1116-1135 (October 2017)). These mutations included Isocitrate Dehydrogenase (NADP(+)) 1 (IDH1), Isocitrate Dehydrogenase (NADP(+)) 2 (IDH2), BRCA1 Associated Protein 1 (BAP1), Fibroblast Growth Factor Receptor 2 (FGFR2), Kirsten Rat Sarcoma Viral Oncogene Homologue (KRAS), Polybromo 1 (PBRM1), AT-Rich Interaction Domain 1A (ARID1A), Phosphatidylinositol-4,5-Bisphosphate 3-Kinase Catalytic Subunit Alpha (PIK3CA), Ephrin type-A receptor 2 (EPHA2), Cyclin-Dependent Kinase Inhibitor 2A (CDKN2A), Tumor Protein P53 (TP53), SMAD Family Member 4 (SMAD4), and Transforming Growth Factor Beta Receptor 2 (TGFBR2).

Comprehensive computational analysis revealed that CLDN1 is robustly expressed across different CCA genetic mutations (FIG. 1D). CLDN1 expression is highest in tumors with IDH1 and BAP1 genetic mutations compared to other mutations (GSE89747 and GSE89748, **p<0.01, Mann-Whitney test, FIG. 1D). CLDN1 expression was lowest in TP53, SMAD4 and TGFBR2 mutations compared to other mutations (GSE89747 and GSE89748, **p<0.01. *p<0.05. Mann-Whitney test, FIG. 1D). The mutation frequencies of driver genes differ between CCA etiologies, with BAP1 and IDH1 mutations being highly enriched in non-fluke-related CCAs, and TP53 and SMAD4 being highly enriched in fluke-related CCAs (Brindley et al., 7(65) Nat Rev Dis Primers (September 2021)).

Taken together, up-regulation of CLDN1 expression in both intrahepatic and extrahepatic CCA tumors underscored its potential involvement in CCA disease pathogenesis and outcome and suggests that CLDN1 is a therapeutic target for treatment of CCA.

### Example 2. CLDN1 mAb H3L3 Suppresses Tumor Growth in Both Cell Line-Derived Xenograft (CDX) and Patient-Derived Xenograft (PDX) Mouse Models

To assess the in vivo anti-tumorigenic activity of an anti-CLDN1 monoclonal (CLDN1 mAb H3L3) antibody in CCA, anti-CLDN1 mAb's effect on tumor growth was investigated in both intrahepatic CCA and extrahepatic CCA cell line-derived xenograft (CDX) mouse models.

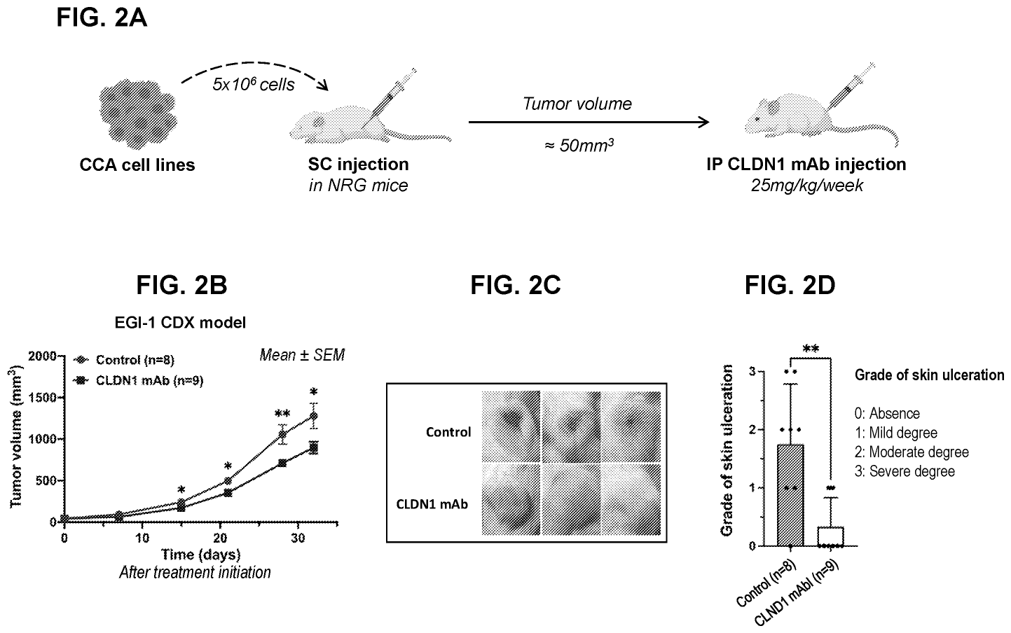

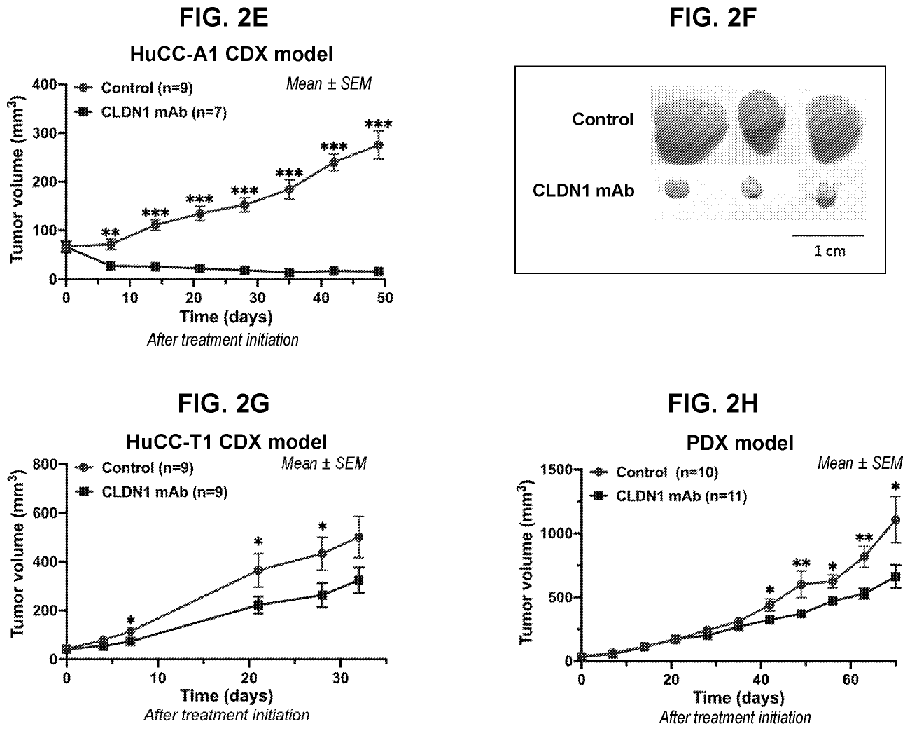

Non-Obese Diabetic Rag1^{-/-}IL2Rgc^{-/-} (NRG) were subcutaneously injected with 5×10⁶ cells of HuCC-A1 (intrahepatic CCA) or EGI-1 (extrahepatic CCA) cell line. Non-Obese Diabetic Prkdc^{scid}IL2Rg^{-/-} (NSG) were subcutaneously injected with 5×10⁶ cells of the HuCC-T1 (intrahepatic CCA) cell line. When the average tumor volumes reached 40-50 mm³, mice were randomized into two different groups and treated for 6 weeks with the anti-CLDN1 mAb H3L3 (25 mg/kg i.p. once per week), or with vehicle control (PBS i.p. once per week). Tumor growth was monitored using a digital caliper and tumor volume was calculated using the ½ (length×width²) formula. (FIG. 2A).

CLDN1 mAb H3L3 significantly reduced tumor growth in the three CDX models (FIGS. 2B-2G). Strong and robust inhibition of tumor growth over time was noted in HuCC-A1 CDX mice (p<0.001, Mann-Whitney test), highly reflected by the macroscopic appearance of the explanted tumors (FIGS. 2E-2F). Moreover, the antibody delayed tumor growth of EGI-1 CDX (p<0.05, Mann-Whitney test, FIG. 2B) and HuCC-T1 CDX mice (p<0.05, Mann-Whitney test, FIG. 2G). In addition, anti-CLDN1 mAb H3L3 demonstrated a significant reduction of skin ulceration induced by tumors in the EGI-1 model (p<0.01, Mann-Whitney test, FIGS. 2C-2D), suggesting an effect on tumor cell invasion.

On the other hand, molecular drivers and treatment responses strongly vary between different CCA subclasses and patients (Banales et al., 17 Nat Rev Gastroenterol Hepatol 557-588 (2020)). In this context, PDX mouse models have been shown to partially recapitulate tumoral heterogeneity and are currently one of the most widely used in vivo systems for studying cancer therapeutics and predicting clinical outcomes (Xu et al., 17(1) Oncol Lett 3-10 (January 2019)). Thus, a PDX mouse model was established to evaluate the anti-CLDN1 mAb H3L3 anti-tumoral efficacy and response rate.

Non-Obese Diabetic Rag1^{-/-}IL2Rgc^{-/-} (NRG) were subcutaneously injected with fresh tumor tissues cut into small pieces (approximately 2-3 mm in diameter). When the average tumor volumes reached 40 mm³, mice were randomized into two different groups and treated with the anti-CLDN1 mAb H3L3 (25 mg/kg i.p. once per week, n=10 mice), or with vehicle control (PBS i.p. once per week, n=11 mice). Tumor growth was monitored using a digital caliper and tumor volume was calculated using the ½(length×width²) formula.

In line with the results obtained in CDX mice, treatment with anti-CLDN1 mAb H3L3 markedly and significantly slowed tumor growth in the PDX model (p<0.05, Mann-Whitney test, FIG. 2H). Altogether, these data denoted the anti-tumoral effects of non-junctional CLDN1-targeting mAb in both cell line-derived and patient-derived tumor models, providing a robust preclinical proof-of-concept of CLDN1 mAbs for CCA treatment.

### Example 3. Anti-CLDN1 Antibodies Inhibit Migration and Invasion of CCA Cell Lines

Next, the effect of anti-CLDN1 mAb H3L3 on cell migration was assessed in a cell-based wound healing assay using an extrahepatic CCA cell line (KKU100), and an intrahepatic CCA cell line (HuCC-A1).

In the wound healing assay, 1.5×10⁵ CCA cells were co-cultured in 12 well plates (Corning) with LX2 in a ratio of 1:1 to reproduce the stromal architecture seen in CCA tumors. Co-cultures were treated for 3 days with 20 μg/mL isotype control mAb or with 20 μg/mL anti-CLDN1 mAb H3L3. At day 3, wounds were created in the confluent cell monolayer using a pipette tip. Cells were then washed once with PBS to remove debris and covered with 1% FBS media and treated again with 20 μg/mL isotype control mAb or with 20 μg/mL anti-CLDN1 mAb H3L3. Plates were incubated at 37° C. and wounds were imaged at 0, 24 and 48 h. Relative percent of wound closure was measured using ImageJ. Each experiment was independently repeated at least three times.

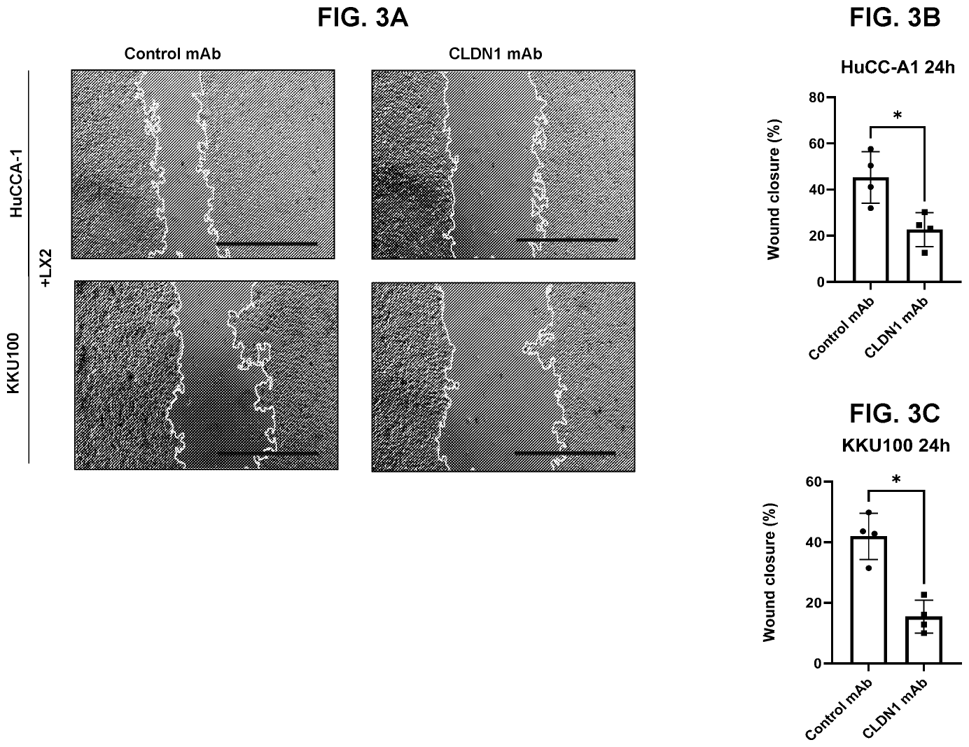

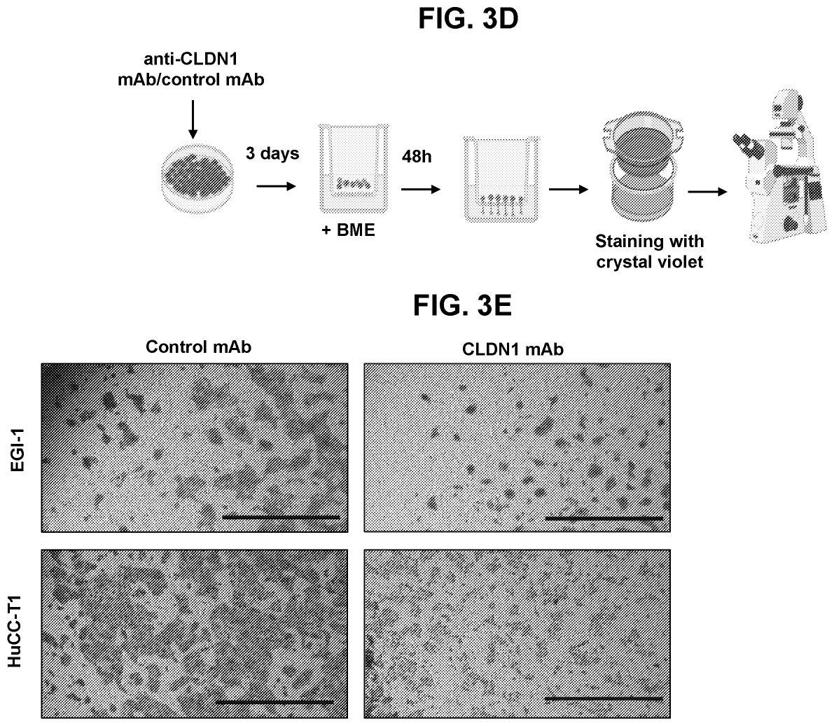

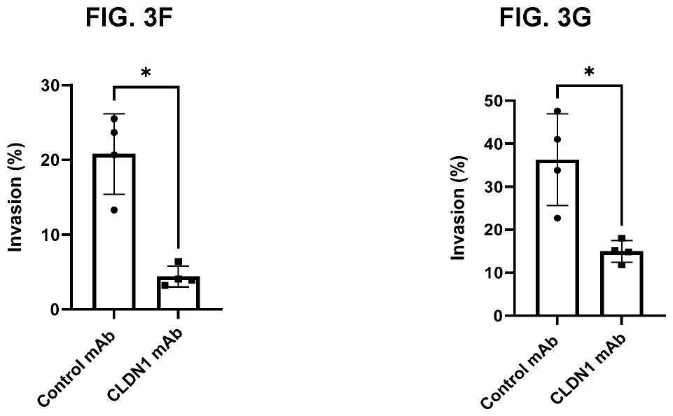

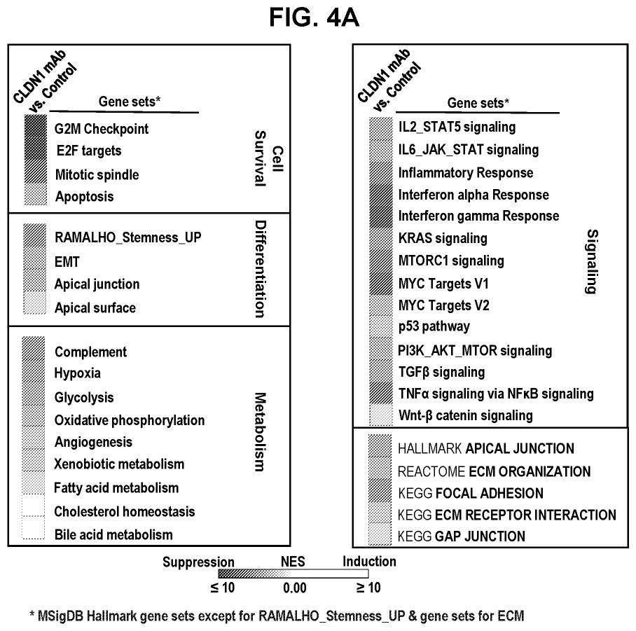

Treatment with anti-CLDN1 mAb H3L3 significantly and specifically decreased CCA cell migration in the wound healing assay (p=0.02, Mann-Whitney test, FIGS. 3A-3C) compared to treatment with an isotype antibody.

In an additional study, a transwell invasion assay was used to assess the effect of anti-CLDN1 mAb H3L3 treatment on cancer invasion. In this assay, EGI-1 or HuCC-T1 cells were seeded in 12 well plates and treated with 20 μg/mL isotype control mAb or with 20 μg/mL anti-CLDN1 mAb H3L3 (Colpitts et al., 67(4) Gut 736-745 (March 2017)) for 3 days. 0.5×10⁵ cells were then seeded in transwells (8-μm pore polycarbonate membrane insert) in 24-well dishes with 75 μl Matrigel (Corning) and serum-free medium (1:20). Following solidification for 10 minutes in the incubator (37° C., 5% CO₂), 600 μL of 1% serum-supplemented medium was added to the bottom wells. Non-invading cells were removed by scrubbing with cotton swabs, and invading cells were fixed using 70% ethanol and stained with crystal violet (0.2%) after 72 hours. Cells on the underside of the membranes were then visualized in 5 randomly selected fields and quantified using ImageJ. Each experiment was independently repeated at least three times.

Treatment with the anti-CLDN1 mAb H3L3 markedly and significantly decreased the number of invasive EGI-1 and HuCC-T1 cells compared to isotype control antibody-treated cells (p=0.02, Mann-Whitney test, FIGS. 3D-3G).

Collectively, these data demonstrate that treatment with anti-CLDN1 mAbs inhibited the migratory and invasive abilities of CCA cell lines further confirming the anti-tumor effect of anti-CLDN1 antibodies.

### Example 4. CLDN1 mAb Mediates Anti-Tumorigenic Effects by Interfering with Cancer Cell Differentiation, Metabolism, and Oncogenic Pathways

To elucidate the molecular mechanisms underlying the anti-tumor effect provided by anti-CLDN1 mAb (H3L3), RNA-sequencing (RNA-seq) was performed on the HuCC-A1 tumor tissue from CDX mice.

Gene Set Enrichment Analysis (GSEA) was used for unbiased pathway analysis using the Molecular Signature Database (MSigDB v. 7.4). For evaluation of anti-CLDN1 mAb H3L3 treatment effects in the HuCC-A1 CDX mouse model, samples derived from anti-CLDN1 H3L3 mAb treated animals (n=5 mice) were compared to respective control samples of the same model (n=5 mice). Results from GSEA were adjusted for the false discovery rate (FDR). An FDR<0.05 was considered statistically significant.

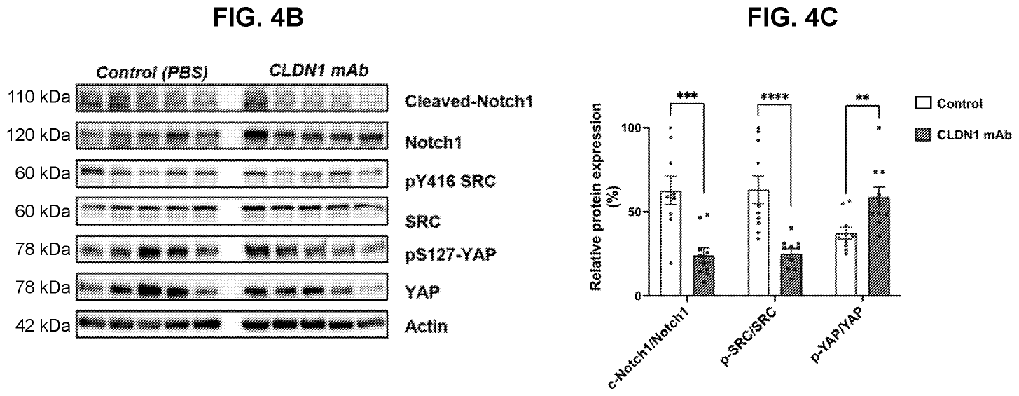

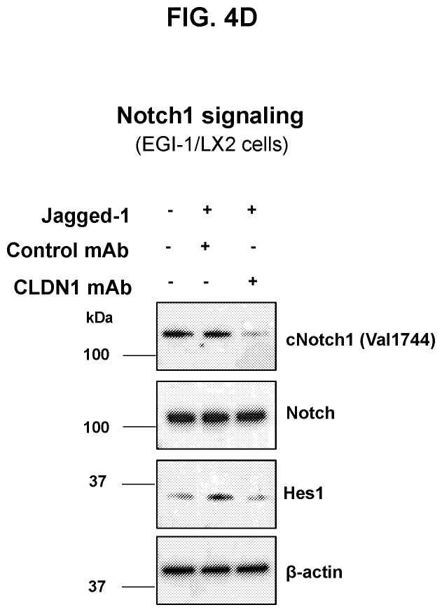

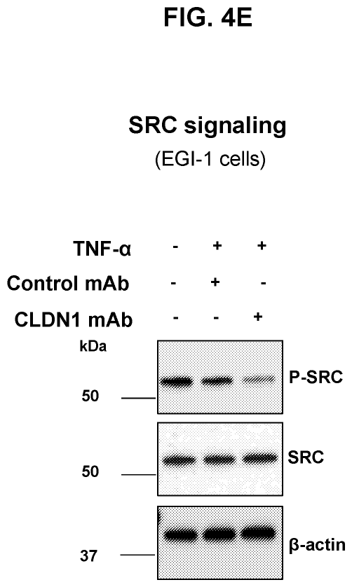

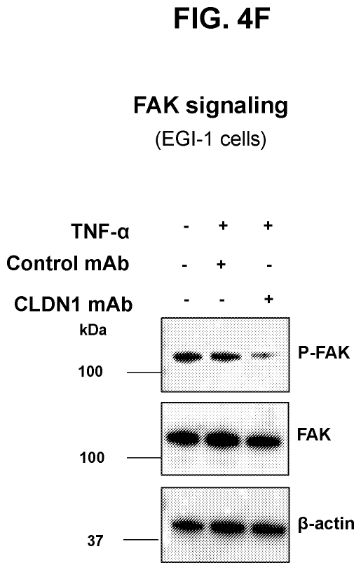

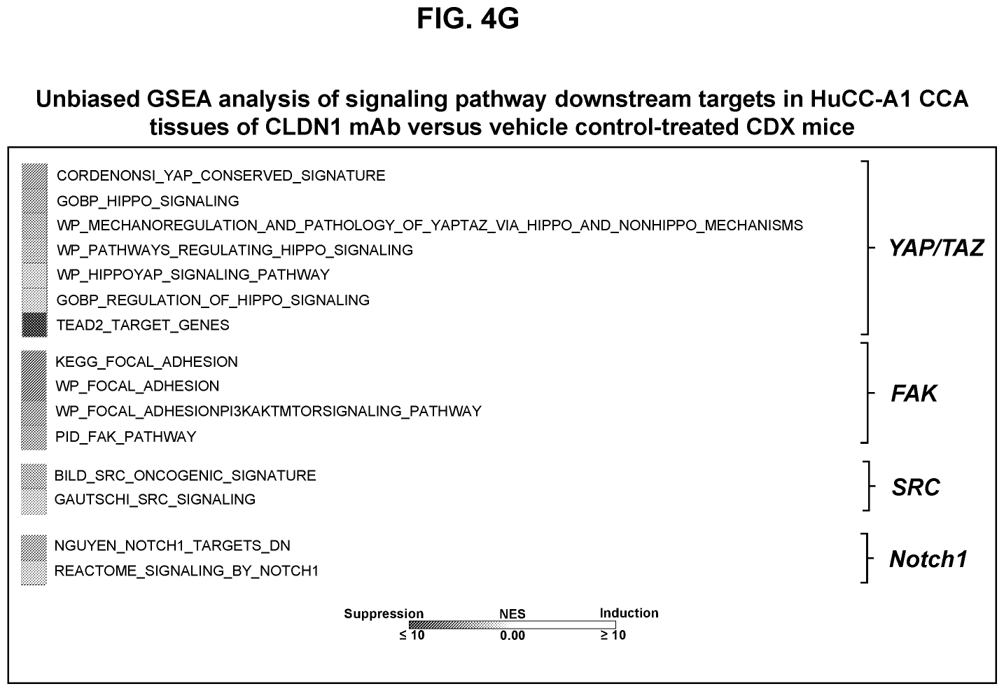

Gene sets associated with cell survival, metabolism and differentiation, including EMT were markedly downregulated in CLDN1 mAb H3L3-treated mice (FIG. 4A, left panels). In addition to the downregulation of these pathways, targeting non-junctional CLDN1 showed strong suppression of several key oncogenic signaling pathways, with the strongest effects being noted on TNF-α/NF-κB, TGF-β, IL-6/JAK/STAT3, and PI3K/Akt/mTOR signaling (FIG. 4A, right panels), highlighting the major role of non-junctional CLDN1 in driving carcinogenic signaling.

To further investigate the antitumoral effect of CLDN1-targeting mAb, key signaling pathways implicated in CCA development and progression were analyzed in EGI-1 CDX tumor samples (FIGS. 4B-4C).

For protein extraction from CDX tumors, tumor pieces were lysed with lysis buffer (Triton 1%; NaCl 50 mM; Tris 50 mM pH 7.6; MgCl2 2 mM in ddH2O) supplemented with proteinase inhibitors (Roche) and phosphatase inhibitors (Sigma Phosphatase Cocktail number 2 and 3) during 30 min at 4° C. Then the samples were centrifuged at 10000×g for 10 min and the supernatant was collected. Protein quantification was assessed with Thermo Scientific BCA Kit. Gels were prepared following the Biorad TGX gel protocol (BioRad). The gels were transferred using the Biorad trans blot turbo protocol (BioRad) into PVDF membranes. Blocking of the membranes was performed with 5% BSA in TBS-T for 1 h. The membranes were incubated with YAP/TAZ (D24E4) Rabbit mAb (#8418, Cell Signaling Technology), Phospho-YAP (Ser127) (D9W2I) Rabbit mAb (#13008, Cell Signaling Technology), SRC Rabbit mAb (#21085, Cell Signaling Technology), Phospho-SRC (Tyr416) Rabbit mAb (#21015, Cell Signaling Technology), Notch1 (D1E11) XP® Rabbit mAb (#3608, Cell Signaling Technology), Cleaved Notch1 (Val1744) (D3B8) Rabbit mAb (#4147, Cell Signaling Technology) and monoclonal anti-β-Actin antibody produced in mouse (Sigma) in 2.5% BSA in TBS-T with a dilution of 1:1000 overnight at 4° C. The membranes were incubated with Horseradish Peroxidase conjugated secondary antibodies in 2.5% BSA in TBS-T with a dilution of 1:10000 for 1 h at room temperature. Protein immunodetection of the membranes was performed with Clarity ECL Western Blot Substrate (Biorad) in a ChemiDoc MP Imaging System (Biorad). Immunoblot images were analyzed using Image Lab Software v6.1 (Biorad).

Western blot quantification analyses revealed a reduction of cleaved Notch1 in anti-CLDN1 mAb H3L3-treated tumors compared to the control one (p<0.001, Mann-Whitney test, FIG. 4C), known to promote CCA cell proliferation and survival (Singrang et al., 13(2) J Cell Commun Signal 245-254 (June 2019)). Moreover, activation of the non-receptor tyrosine kinase protein SRC was strongly reduced in anti-CLDN1 mAb H3L3-treated tumors, indicated by a significant decrease in SRC phosphorylation (p<0.0001, Mann-Whitney test, FIG. 4C). The Yes-associated protein (YAP)/Hippo pathway was also impacted by anti-CLDN1 antibody treatment. Indeed, a strong increase of YAP S127 phosphorylation was observed upon anti-CLDN1 mAb H3L3 treatment (p<0.01, Mann-Whitney test, FIG. 4C), which leads to YAP cytoplasmic sequestration and inactivation (Sugihara et al., 54(6) J Gastroenterol 485-491 (June 2019)).

Collectively, the results in Examples 1-4 identified non-junctional CLDN1 as a oncogenic driver and therapeutic target in CCA. Targeting CLDN1 in perturbation studies using highly specific antibodies showed significant and robust suppression of tumor growth in all tested human CDX and PDX mouse models, as well as inhibition of invasion and migration in state-of-the-art human CCA cell-based models. Mechanistic studies based on RNA-Seq and proteomic analyses on signaling, revealed that this anti-tumorigenic effect was likely mediated through interference with cancer cell differentiation, metabolism, and key oncogenic pathways implicated in CCA pathogenesis.

Lack of response to pharmacological treatment is a major limitation in the management of advanced CCA. Mechanisms of chemoresistance (MOC) are diverse and dependent on the expression and/or function of the MOC genes molecular targets (Marin et al., 1864 (4 Pt B) Biochim Biophys Act Mol Basis Dis 1444-1453 (April 2018)). For example, downregulation of the human equilibrative nucleoside transporter 1 and the copper transporter CTR1 lead to reduced sensitivity toward gemcitabine and cisplatin respectively, the first-line standard-of-care chemotherapies for CCA (Borbath et al., 48(7) Eur J Cancer 990-996 (May 2012)). Advantageously, the unique mechanism of action of CLDN1 mAbs will constitute a vital therapeutic option to circumvent the multidrug intrinsic or acquired resistant phenotype in CCA.

Combined hepatocellular and cholangiocarcinoma (HCC-CC) is an aggressive biphenotypic primary liver cancer with unmet needs and unsatisfactory outcomes (Azizi et al., 10 Front Oncol 570958 (September 2020)). Despite recent advances lately, standard first-line systemic therapy options are not well-established, and the surgical approach is the only available curative treatment (Leoni et al., 12(4) Cancers (Basel) 794 (March 2020)). Claudin-1 may advantageously provide a dual-targeting therapeutic target also for HCC-CC.

The complex interplay between various signal transduction pathways orchestrating cholangiocarcinogenesis and mediating tumor development has been delineated in the past decades (Banales et al., 17 Nat Rev Gastroenterol Hepatol 557-588 (2020)). Among others, highly conserved pathways, such as YAP, the Hippo pathway effector protein, and Notch are prominently active in CCA with an upregulation of 67.2% and 79.5% in CCA tumors, respectively (Wu et al., 37(10) Tumour Biol. 13499-13508 (October 2016); Wu et al., 31(6) Oncol Rep 2515-2524 (June 2014)). The data demonstrated a strong downregulation of Notch1 and YAP signaling upon the anti-CLDN1 mAb H3L3 treatment (FIG. 4B-4C). By targeting two independent, yet interconnected signaling pathways, anti-CLDN1 mAbs may circumvent the acquired therapeutic resistance encountered by previous targeted therapy against CCA (Simile et al., 55(2) Medicina (Kaunas) 42 (February 2019)). Furthermore, targeting SRC, a central component upstream of multiple transduction pathways (Ishizawar et al., 6(3) Cancer Cell 209-214 (September 2004)), may reduce the activation of compensatory signaling in single targeting approaches due to functional cross-talks and redundancy in the maintenance of key pathways in this disease. Taken together, this unique mechanism of action uncovers a novel therapeutic approach to tackle the inter- and intra-tumoral heterogenous properties deregulating distinct signaling cascades in different subsets of CCAs.

To corroborate the effect of the anti-CLDN1 mAb H3L3 on CCA signaling observed in vivo (FIGS. 4A-4C), additional signaling studies were performed in CCA cell-based models. The CCA models consisted of 1.5×10⁵ EGI-1 CCA cells or a co-culture of CCA EGI-1 with LX2 stellate cells (ratio 1:5) treated with anti-CLDN1 mAb H3L3 (Colpitts et al., 67(4) Gut 736-745 (March 2017)) (10 μg/mL) or isotype control mAb (10 μg/mL) for 2 days. At day 2 post-treatment, cells were stimulated with TNF-α (10 ng/mL) for 24 hours to assess SRC and FAK signaling, or Jagged-1 (50 ng/mL, SRP8012, Sigma-Aldrich) for 24 hours to assess Notch1 signaling. For protein extraction from cell cultures, cells were lysed using Glo lysis buffer (E2661, Promega) supplemented with protease and phosphatase inhibitors (78442, Halt™ Protease and Phosphatase Inhibitor Single-Use Cocktail, Thermo Fisher) for 20 minutes at 4° C. Samples were then centrifuged at 15000 rpm for 10 minutes and the supernatant was collected. Protein quantification was performed using the Bio-Rad DC assay Kit. Gels were prepared following the Biorad TGX gel protocol (BioRad). Gels were transferred using the Biorad trans blot turbo protocol (BioRad) into PVDF membranes. Blocking of the membranes was performed with 5% BSA in TBS-T for 1 hour. The membranes were incubated with anti-SRC Rabbit mAb (#21085, Cell Signaling Technology), Phospho-SRC (Tyr416) Rabbit mAb (#21015, Cell Signaling Technology), anti-Notch1 (D1E11) XP® rabbit mAb (#3608, Cell Signaling Technology), anti-cleaved Notch1 (Val1744) (D3B8) rabbit mAb (#4147, Cell Signaling Technology), anti-FAK rabbit mAb (#3285, Cell Signaling Technology), anti-phospho-FAK (Tyr576/577) rabbit mAb (#3281, Cell Signaling Technology) and anti-β-actin antibody (Sigma) in 5% milk in TBS-T for 2 hours at room temperature. Secondary antibodies used were peroxidase AffiniPure goat anti-rabbit IgG (H+L) mAb (Jackson ImmunoResearch) or ECL mouse IgG, HRP-linked whole Ab (Amersham). Protein immunodetection of the membranes was performed with Clarity ECL Western Blot Substrate (Biorad) in a ChemiDoc MP Imaging System (Biorad). Each experiment was independently repeated at least three times.

Western blot analyses of the CCA cell line EGI-1 treated with anti-CLDN1 mAb H3L3 or isotype control mAb showed a robust decrease in cleaved Notch1 and its downstream target Hes1 (FIG. 4D), confirming that treatment with the anti-CLDN1 mAb inhibits Notch1 signaling in CCA cells. In addition, SRC and FAK phosphorylation were reduced upon treatment with the anti-CLDN1 mAb compared to control-treated cells (FIGS. 4E-4F), confirming the effect of anti-CLDN1 mAb on those signaling pathways in CCA cells. Since Notch1, SRC and FAK pathways have been identified as key signaling pathways driving CCA development and progression (Banales et al., 17(9) Nat Rev Gastroenterol Hepatol 557-588 (September 2020)), these data indicate that inhibition of Notch1, SRC and FAK signaling by anti-CLDN1 mAb contribute to the anti-tumor effects of the antibodies.

To further understand and confirm the effect of anti-CLDN1 mAb treatment on the identified oncogenic signaling pathways, gene set enrichment analysis (GSEA) was applied for unbiased pathway analysis using the Molecular Signature Database (MSigDB v. 7.4) in the HuCC-A1 CDX mouse tumors. Samples derived from anti-CLDN1 mAb H3L3 treated animals (n=5 mice) were compared to respective control samples of the same model (n=5 mice). Results from GSEA were adjusted for the false discovery rate (FDR). An FDR<0.05 was considered statistically significant.

Expression of the downstream target and effector genes of YAP/TAZ, FAK, SRC and Notch1 signaling pathways were markedly downregulated in the anti-CLDN1 mAb H3L3-treated group compared to the control (FIG. 4G), confirming the functional relevance of the identified pathways in anti-CLDN1 mAb treated CCAs.

Collectively, these results provided robust pre-clinical proof-of-concept of the therapeutic effect of CLDN1-specific monoclonal antibodies and unravel anti-CLDN1 mAbs as an innovative therapeutic modality for the treatment of CCA, a rapidly growing unmet medical need worldwide with unsatisfactory treatment options.

### Example 5. In Vivo Inhibition of Lung Metastasis by Treatment with CLDN1 mAb in a Mouse Model for CCA Metastasis

To assess the in vivo activity of the anti-CLDN1 mAb (H3L3) on invasion and metastasis, treatment with the anti-CLDN1 antibody was investigated in a state-of-the-art mouse model for CCA metastasis based on intravenous injection of metastatic HuCC-T1 CCA cells.

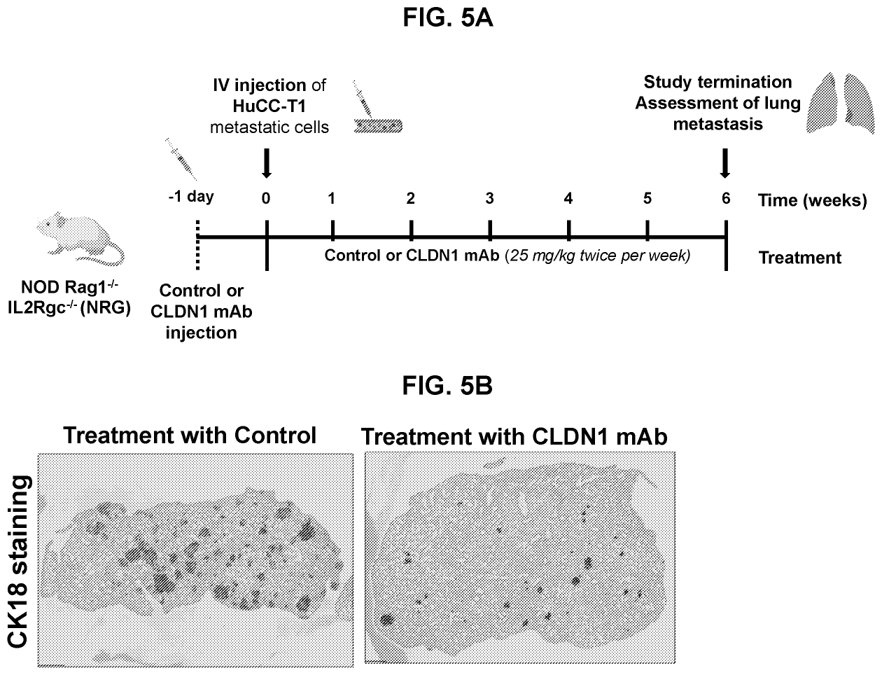

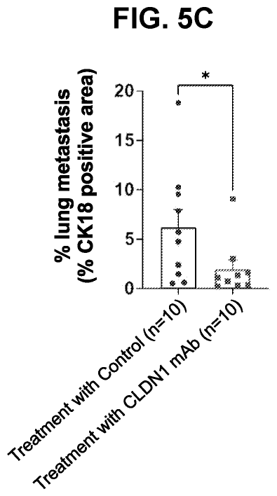

Non-Obese Diabetic Rag1^{-/-}IL2Rgc^{-/-} (NRG) mice were treated with anti-CLDN1 mAb H3L3 (Colpitts et al., 67(4) Gut 736-745 (March 2017)) (25 mg/kg i.p.), or vehicle control (PBS i.p.) 1 day before intravenous injection of 0.5×10⁶ h HuCC-T1 cells through the retro-orbital vein. Mice were randomized into two groups and treated for 6 weeks with anti-CLDN1 mAb H3L3 (Colpitts et al., 67(4) Gut 736-745 (March 2017)) (25 mg/kg i.p. twice per week) or vehicle control (PBS i.p. twice per week). After 6 weeks, mice were sacrificed, lungs were harvested, and cytokeratin 18 (CK18) staining was used to identify and quantify CK18-positive cholangiocarcinoma metastasis in the lungs (FIG. 5A).

Anti-CLDN1 mAb H3L3 significantly reduced metastasis formation in lungs compared to the control group as demonstrated by a decrease in CK18 positive area upon treatment with anti-CLDN1 mAb H3L3 in lung sections (p<0.05, Mann-Whitney test) (FIGS. 5B-5C). These data show that anti-CLDN1 mAb H3L3 treatment results in the inhibition of CCA metastasis formation in vivo.

### Example 6. CLDN1 Expression Targeted by CLDN1 mAb H3L3 is Upregulated in CCA Upon Treatment with Cisplatin and Gemcitabine

A first-line chemotherapy treatment for advanced CCA is cisplatin plus gemcitabine (Borbath et al., 48(7) Eur J Cancer 990-996 (May 2012)). To investigate the expression of CLDN1 upon treatment with cisplatin and gemcitabine, 1.5×10⁵ CCA EGI-1 cells were treated with cisplatin (100 nM) and gemcitabine (10 nM) for 24 hours. Cells were then collected, and CLDN1 expression was assessed by flow cytometry and Western Blot using CLDN1 specific Abs.

For flow cytometry, cells were incubated with the anti-CLDN1 mAb H3L3 or the isotype control mAb at a concentration of 10 μg/ml in triplicates per condition. Following incubation with the primary antibody for 1 hour, cells were washed and incubated with human Alexa647-conjugated secondary antibody at 4° C. for 45 minutes. Cells were subsequently washed and fixed with 2% paraformaldehyde (PFA). Data were acquired using Cytoflex B2R2V0 (Beckman Coulter) and analyzed using CytExpert 2.1 and FlowJo v10 (Beckman Coulter). CLDN1 expression was calculated as the difference of the mean fluorescence intensities of cells stained with anti-CLDN1 mAb H3L3 and isotype control mAb. The experiment was independently repeated three times.

For protein extraction for Western Blot analyses, cells were lysed using Glo lysis buffer (E2661, Promega) supplemented with protease and phosphatase inhibitors (78442, Halt™ Protease and Phosphatase Inhibitor Single-Use Cocktail, Thermo Fisher) for 20 minutes at 4° C. Samples were then centrifuged at 15000 rpm for 10 minutes and the supernatant was collected. Protein quantification was performed using the Bio-Rad DC assay Kit. Gels were prepared following the Biorad TGX gel protocol (BioRad). Gels were transferred using the Biorad trans blot turbo protocol (BioRad) into PVDF membranes. Blocking of the membranes was performed with 5% BSA in TBS-T for 1 hour. The membranes were incubated with anti-CLDN1 Rabbit mAb (#E-AB-15674, ElabScience) and anti-β-actin antibody (Sigma) in 5% milk in TBS-T for 2 hours at room temperature. Secondary antibodies used were peroxidase AffiniPure goat anti-rabbit IgG (H+L) mAb (Jackson ImmunoResearch) or ECL mouse IgG, HRP-linked whole Ab (Amersham). Protein immunodetection of the membranes was performed with Clarity ECL Western Blot Substrate (Biorad) in a ChemiDoc MP Imaging System (Biorad). The experiment was independently repeated at least three times.

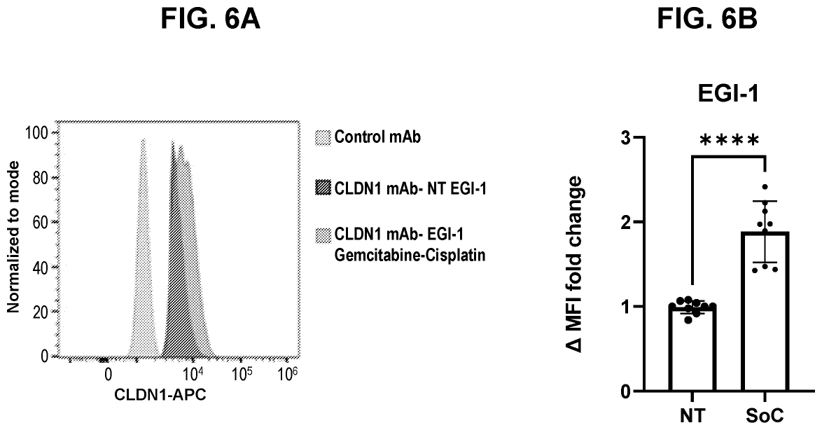

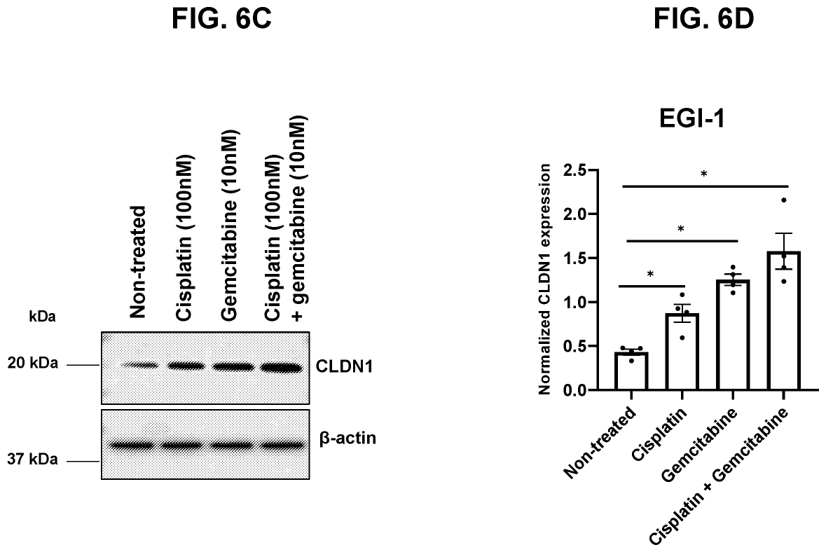

Flow cytometry analyses of the CCA cell line EGI-1 treated with cisplatin and gemcitabine using anti-CLDN1 mAb H3L3 or control Abs showed a robust increase in CLDN1 expression upon treatment with cisplatin and gemcitabine (FIGS. 6A-6B). This upregulation was further confirmed by Western Blot analyses using CLDN1-specific Abs (FIGS. 6C-6D).

These data show that chemotherapy with cisplatin and gemcitabine results in the upregulation of exposed and targetable CLDN1 on the CCA cell surface which is detected and accessible by anti-CLDN1 mAb H3L3. This increase in CLDN1 expression in chemotherapy-treated CCA offers the opportunity to treat CCA with anti-CLDN1 mAbs in patients in combination or following treatment with chemotherapy including cisplatin and gemcitabine to improve patients' outcome.

### Example 7. Anti-CLDN1 mAb H1L1 Inhibits Tumor Growth in a Cholangiocarcinoma PDX Model

The objective of this experiment was to evaluate preclinically the in vivo therapeutic efficacy of the anti-CLDN1 mAb H1L1 in the treatment of a subcutaneously PDX models for cholangiocarcinoma (CC6702 model) in female BALB/c nude mice. The animal number was set to n=2 in Placebo and n=3 in the treatment group.

Fresh tumor tissues from mice bearing established PDX model were harvested and cut into small pieces (approximately 2-3 mm in diameter). PDX tumor fragments, harvested from donor mice, were inoculated subcutaneously at the upper right dorsal flank into study mice for tumor development. The randomization started when the mean tumor size reached approximately 100-150 mm³. A total of 5 mice per model were enrolled in the study and allocated into 2 groups. Randomization was performed based on “Stratified” method (Study Director™ software, version 3.1.399.19). The date of grouping was denoted as day 0.

For the control group, 0.2 mL of placebo was administered via i.p. injection (QW×4). For the treatment group, 0.2 mL of anti-CLDN1 mAb H1L1 (2.5 mg/mL) was administered via i.p. injection (QW×4).

After tumor inoculation, the animals were checked daily for morbidity and mortality. During routine monitoring, the animals were checked for any effects of tumor growth and treatments on behavior such as mobility, food and water consumption, body weight gain/loss (Body weights would be measured twice per week after randomization), eye/hair matting and any other abnormalities. Mortality and observed clinical signs were recorded for individual animals in detail.

Tumor volumes were measured twice per week after randomization in two dimensions using a caliper, and the volume was expressed in mm3 using the formula: V=(L×W×W)/2, where V is tumor volume, L is tumor length (the longest tumor dimension) and W is tumor width (the longest tumor dimension perpendicular to L). Dosing as well as tumor and body weight measurements were conducted in a Laminar Flow Cabinet. The body weights and tumor volumes were measured by using Study Director™ software (version 3.1.399.19).

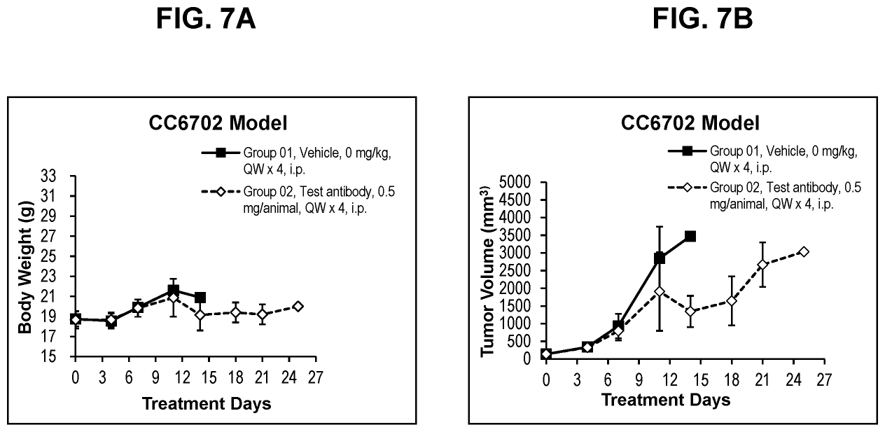

FIG. 7A shows the body weight curves at different time points for the CC6702 PDX model after treatment with vehicle or with anti-CLDN1 mAb H1L1. FIG. 7B shows the tumor volume growth curves at different time points for the CC6702 PDX model after treatment with vehicle or with anti-CLDN1 mAb H1L1. The control group was terminated after day 14 due to humane considerations resulting from too strong tumor growth. FIG. 7B establishes that anti-CLDN1 mAb H1L1 inhibited tumor growth in a cholangiocarcinoma PDX model relative to the control-treated group.

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature.

All of the references cited above, as well as all references cited herein, are incorporated herein by reference in their entireties.

Any examples provided herein are offered by way of illustration and not by way of limitation.

1. A method of treating a cholangiocarcinoma (CCA) in a human subject in need thereof, comprising administering a therapeutically effective amount of an anti-Claudin-1 antibody to the human subject, wherein the anti-Claudin-1 antibody comprises a heavy chain variable domain complementary determining region (CDR) H1 comprising the amino acid sequence set forth in SEQ ID NO: 5, a CDR H2 comprising the amino acid sequence set forth in SEQ ID NO: 6, and a CDR H3 comprising the amino acid sequence set forth in SEQ ID NO: 7, and a light chain variable domain complementary determining region (CDR) L1 comprising the amino acid sequence set forth in SEQ ID NO: 8, a CDR L2 comprising the amino acid sequence GAS, and a CDR L3 comprising the amino acid sequence set forth in SEQ ID NO: 10.

2. The method of claim 1, wherein the CCA is an intrahepatic CCA.

3. The method of claim 1, wherein the CCA is a perihilar CCA.

4. The method of claim 1, wherein the CCA is a distal CCA.

5. The method of claim 1, wherein the CCA is a combined or mixed hepatocellular cholangiocarcinoma (cHCC-CCA).

6. The method of claim 1, wherein Claudin-1 (CLDN1) is overexpressed in the human subject compared to expression levels in a normal subject.

7. (canceled)

8. (canceled)

9. The method of claim 1, wherein the anti-Claudin-1 antibody is humanized.

10. The method of claim 1, wherein the anti-Claudin-1 antibody comprises a VH comprising the amino acid sequence set forth in SEQ ID NO: 3 or SEQ ID NO: 13.

11. The method of claim 1, wherein the anti-Claudin-1 antibody comprises a VL comprising the amino acid sequence set forth in SEQ ID NO: 4 or SEQ ID NO: 14.

12. The method of claim 1, wherein the anti-Claudin-1 antibody comprises a VH comprising the amino acid sequence set forth in SEQ ID NO: 3; and a VL comprising the amino acid sequence set forth in SEQ ID NO: 4.

13. The method of claim 1, wherein the anti-Claudin-1 antibody comprises a VH comprising the amino acid sequence set forth in SEQ ID NO: 13; and a VL comprising the amino acid sequence set forth in SEQ ID NO: 14.

14. The method of claim 1, wherein the anti-Claudin-1 antibody comprises a heavy chain comprising the amino acid sequence set forth in SEQ ID NO: 1 or SEQ ID NO: 15; and a light chain comprising the amino acid sequence set forth in SEQ ID NO: 2.

15. The method of claim 1, wherein the anti-Claudin-1 antibody is administered intratumorally, intravenously, intraperitoneally, intramuscularly, intrathecally or subcutaneously.

16.-30. (canceled)

31. The method of claim 1, wherein the CCA contains one or more genetic mutations in one of more of the following genes: Isocitrate Dehydrogenase (NADP(+)) 1 (IDH1), Isocitrate Dehydrogenase (NADP(+)) 2 (IDH2), BRCA1 Associated Protein 1 (BAP1), Fibroblast Growth Factor Receptor 2 (FGFR2), Kirsten Rat Sarcoma Viral Oncogene Homologue (KRAS), Polybromo 1 (PBRM1), AT-Rich Interaction Domain 1A (ARID1A), Phosphatidylinositol-4,5-Bisphosphate 3-Kinase Catalytic Subunit Alpha (PIK3CA), Ephrin type-A receptor 2 (EPHA2), Cyclin-Dependent Kinase Inhibitor 2A (CDKN2A), Tumor Protein P53 (TP53), SMAD Family Member 4 (SMAD4), Transforming Growth Factor Beta Receptor 2 (TGFBR2).

32. The method of claim 1, further comprising administering a chemotherapeutic drug to the human subject in need thereof.

33. The method of claim 32, wherein the chemotherapeutic agent is gemcitabine.

34. The method of claim 32, wherein the chemotherapeutic agent is cisplatin.

35. (canceled)

36. (canceled)

37. The method of claim 1, wherein the CCA is metastatic.
